(19) United States
(12) Patent Application Publication
(10) Pub. No.: US 2023/0137023 A1
(43) Pub. Date: May 4, 2023

(54) PROCESS FOR PREPARING S-BEFLUBUTAMID BY RESOLVING 2-BROMOBUTANOIC ACID

(71) Applicant: Cheminova A/S, Philadelphia, PA (US)

(72) Inventors: Richard M. CORBETT, West Chester, PA (US); Ravindra V. DATAR, Bangalore (IN); Indrajeet M. JAMANE, Bharuch (IN); Jianhua MAO, Shanghai (CN); Shaileshkumar K. PATEL, Bharuch-East (IN); Dongjie PENG, Shanghai (CN)

(21) Appl. No.: 17/798,852

(22) PCT Filed: Feb. 10, 2021
(86) PCT No.: PCT/IB2021/000076

§ 371 Date: Aug. 10, 2022

Related U.S. Application Data
(60) Provisional application No. 62/972,788, filed on Feb. 11, 2020.

(51) Int. Cl. C07C 211/63; C07C 231/14; C07C 51/347

(52) U.S. Cl. CPC C07C 211/63; C07C 231/14; C07C 51/347

(57) ABSTRACT

Disclosed is a method for preparing compound S-1 (S-1) comprising resolving compound rac-2 (rac-2) with a compound of Formula 3 wherein R¹, R⁴, m and n are as defined in the disclosure.

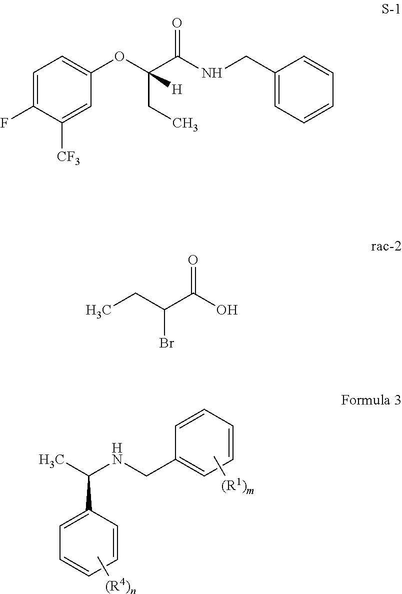

### FIELD OF THE INVENTION

This invention relates to a method for preparing the S-enantiomer of beflubutamid.

### BACKGROUND OF THE INVENTION

U.S. Pat. No. 4,929,273 discloses N-benzyl-2-(4-fluoro-3-trifluoromethylphenoxy)-butanoic amide of Formula 1 as an herbicidal compound. It has a single asymmetric center at the 2-carbon of the amide moiety and thus can be a chiral molecule.

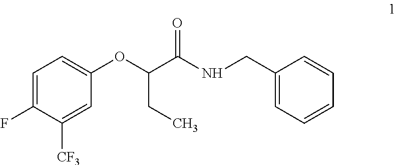 [SMILES: CCC(Oc1ccc(F)c(C(F)(F)F)c1)C(=O)NCc1ccccc1]

This compound in racemic form has been marketed commercially under the common name beflubutamid as a soil herbicide for pre- and post-emergence control of dicotyledonous weeds in cereals. It inhibits the enzyme phytoene-desaturase that is involved in the biosynthesis of carotenoids. Depletion of carotenoids leads to photooxidation of chlorophyll and bleaching/chlorosis of susceptible weeds.

U.S. Pat. No. 4,929,273 also discloses that the (−)-optical isomer is more herbicidally active than the racemic mixture. The more active enantiomer has been identified as having the S-configuration shown as compound S-1 (Environ. Sci. Technol. 2013, 47, 6806-6811 and Environ. Sci. Technol. 2013, 47, 6812-6818).

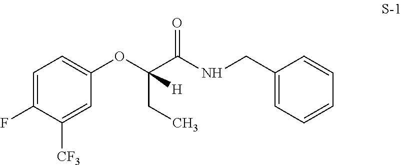 [SMILES: CC[C@H](Oc1ccc(F)c(C(F)(F)F)c1)C(=O)NCc1ccccc1]

While the methods disclosed in the preceding reference can provide the desired compound S-1, continuous improvements are sought, particularly in the development of methods to provide materials on a commercial scale. Therefore, the need continues for new methods that are less costly, more efficient, more flexible, or more convenient to operate.

### SUMMARY OF THE INVENTION

Embodiment A. This invention provides a method for preparing compound S-1

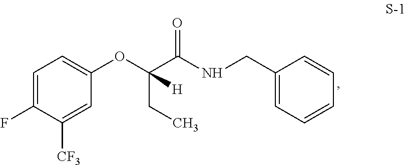 [SMILES: CC[C@H](Oc1ccc(F)c(C(F)(F)F)c1)C(=O)NCc1ccccc1]

from compound R-2

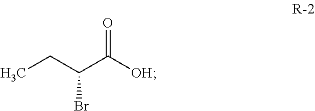

wherein compound R-2 is prepared by
treating compound rac-2

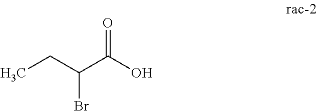

with a compound of Formula 3

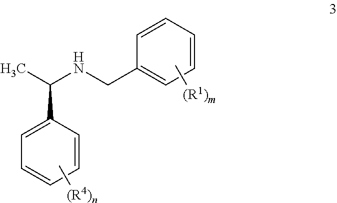

wherein
each R¹ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R²; or
two adjacent R¹ substituents are taken together with the phenyl to which they are attached to form a naphthalenyl ring optionally substituted with up to three R³;
each R² and each R³ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁴ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R⁵;
each R⁵ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
m is 0, 1, 2 or 3; and
n is 0, 1, 2 or 3;
   to provide the R,R-salt of Formula 4

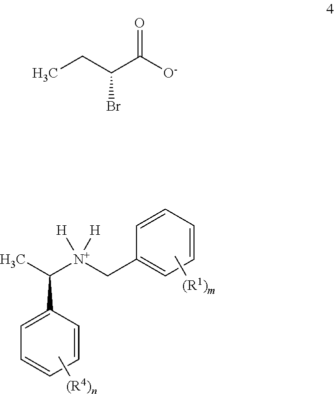

wherein R¹, R⁴, m and n are as defined for the compound of Formula 3; selectively isolating the R,R-salt of Formula 4;
   treating the R,R-salt of Formula 4 with a sodium base to provide compound R-5

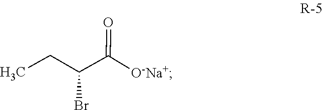

and
treating compound R-5 with acid.

Embodiment B. This invention also provides a method for preparing compound S-1

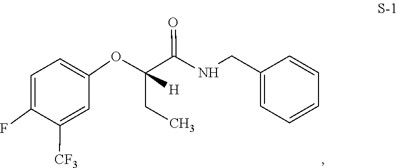 [SMILES: CC[C@H](Oc1ccc(F)c(C(F)(F)F)c1)C(=O)NCc1ccccc1]

the method comprising
preparing compound R-2

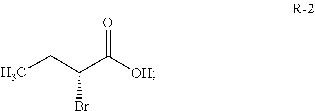

wherein compound R-2 is prepared by
treating compound rac-2

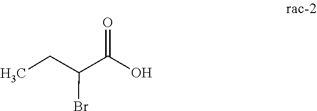

with a compound of Formula 3

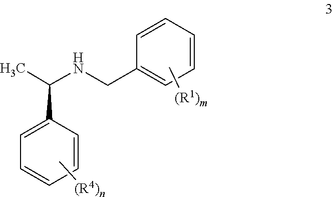

wherein
each R¹ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R²; or
two adjacent R¹ substituents are taken together with the phenyl to which they are attached to form a naphthalenyl ring optionally substituted with up to three R³;
each R² and each R³ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁴ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R⁵;
each R⁵ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
m is 0, 1, 2 or 3; and
n is 0, 1, 2 or 3;
   to provide the R,R-salt of Formula 4

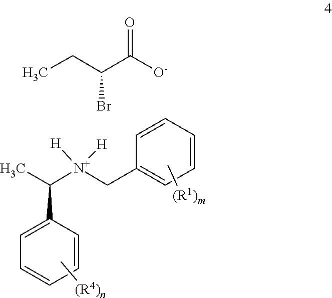

wherein R¹, R⁴, m and n are as defined for the compound of Formula 3; selectively isolating the R,R-salt of Formula 4;
treating the R,R-salt of Formula 4 with a sodium base to provide compound R-5

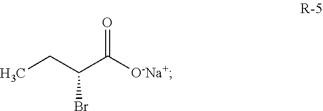

treating compound R-5 with acid; and
converting compound R-2 to compound S-1.

Embodiment C. This invention also provides a method for preparing compound S-1

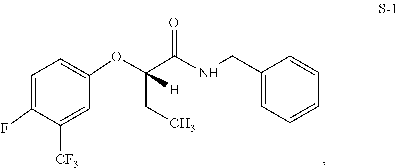 [SMILES: CC[C@H](Oc1ccc(F)c(C(F)(F)F)c1)C(=O)NCc1ccccc1]

the method comprising:
treating compound rac-2

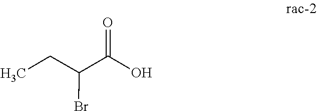

with a compound of Formula 3

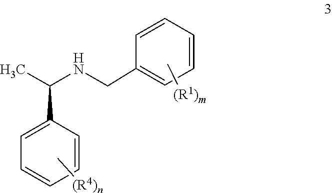

wherein
each R¹ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R²; or
two adjacent R¹ substituents are taken together with the phenyl to which they are attached to form a naphthalenyl ring optionally substituted with up to three R³;
each R² and each R³ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁴ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R⁵;
each R⁵ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
m is 0, 1, 2 or 3; and
n is 0, 1, 2 or 3;
   to provide the R,R-salt of Formula 4

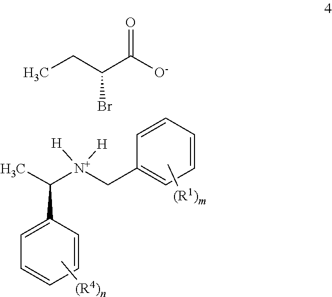

wherein R¹, R⁴, m and n are as defined for the compound of Formula 3;
selectively isolating the R,R-salt of Formula 4;
treating the R,R-salt of Formula 4 with a sodium base to provide compound R-5

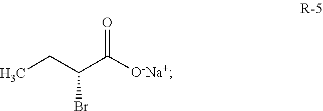

treating compound R-5 with acid to prepare compound R-2

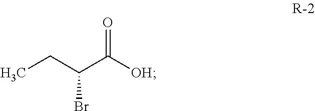

and
converting compound R-2 to compound S-1.

Embodiment D. This invention also provides a method for preparing compound S-1

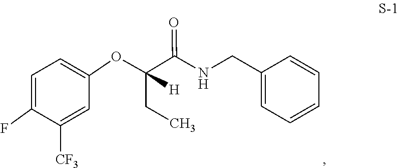 [SMILES: CC[C@H](Oc1ccc(F)c(C(F)(F)F)c1)C(=O)NCc1ccccc1]

the method comprising:
treating compound rac-2

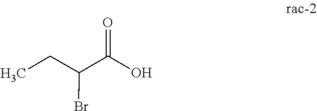

with a compound of Formula 3

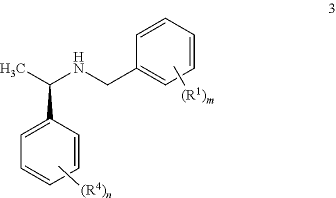

wherein
each R¹ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R²; or
two adjacent R¹ substituents are taken together with the phenyl to which they are attached to form a naphthalenyl ring optionally substituted with up to three R³;
each R² and each R³ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁴ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R⁵;
each R⁵ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
m is 0, 1, 2 or 3; and
n is 0, 1, 2 or 3;
   to provide the R,R-salt of Formula 4

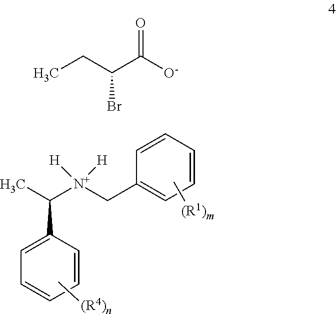

wherein R¹, R⁴, m and n are as defined for the compound of Formula 3; selectively isolating the R,R-salt of Formula 4;
   treating the R,R-salt of Formula 4 with a sodium base to provide compound R-5

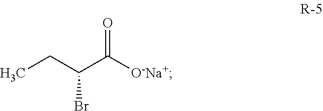

treating compound R-5 with acid to prepare compound R-2

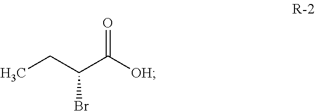

treating compound R-2 with a chlorinating agent to prepare compound R-10

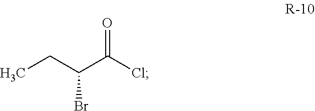

treating compound R-10 with compound 9 (i.e. benzylamine)

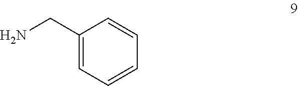

to prepare compound R-11

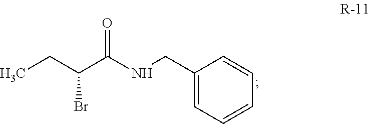

treating compound R-11 with compound 7 (i.e. 4-fluoro-3-(trifluoromethyl)phenol)

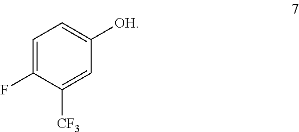 [SMILES: Oc1ccc(F)c(C(F)(F)F)c1]

Embodiment E. This invention also provides a method for preparing compound R-2

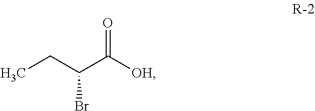

the method comprising:
treating compound rac-2

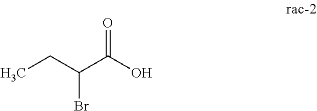

with a compound of Formula 3

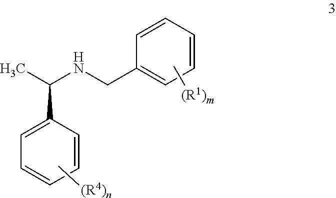

wherein
each R¹ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R²; or
two adjacent R¹ substituents are taken together with the phenyl to which they are attached to form a naphthalenyl ring optionally substituted with up to three R³;
each R² and each R³ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁴ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R⁵;
each R⁵ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
m is 0, 1, 2 or 3; and
n is 0, 1, 2 or 3;
   to provide the R,R-salt of Formula 4

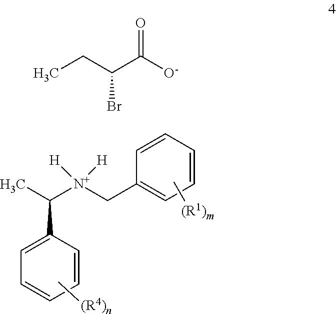

wherein R¹, R⁴, m and n are as defined for the compound of Formula 3; selectively isolating the R,R-salt of Formula 4;
   treating the R,R-salt of Formula 4 with a sodium base to provide compound R-5

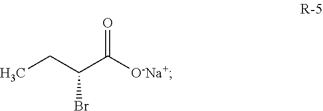

treating compound R-5 with acid.

Embodiment F. This invention also provides a method for preparing compound rac-2

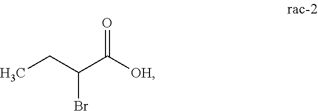

the method comprising:
treating the enantiomerically enriched compound of Formula scal-2

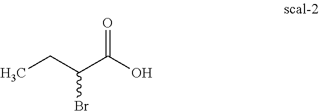

with hydrobromic acid or a quaternary ammonium bromide salt.

Embodiment G. This invention also provides an R,R-salt of Formula 4

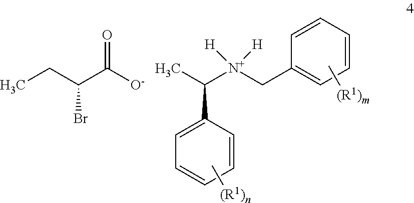

wherein
each R¹ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R²; or
two adjacent R¹ substituents are taken together with the phenyl to which they are attached to form a naphthalenyl ring optionally substituted with up to three R³;
each R² and each R³ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁴ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R⁵;
each R⁵ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
m is 0, 1, 2 or 3; and
n is 0, 1, 2 or 3.

### DETAILED DESCRIPTION OF THE INVENTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, process or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used herein, the term “suitable” indicates that the entity or condition so described is appropriate for use in the situation or circumstance indicated. As used herein, the terms “treatment” or treating” denotes using a chemical or chemical process to alter the existing condition of other materials, chemicals or compounds. The terms “converting,” “converted”, conversion and related words refer to causing an entity such as a chemical compound to change in structure, form, character or function. For example, a compound of a first formula or structure is converted to a compound of a second formula or structure by a chemical process involving one or more treatments as defined above. The term “selectively isolating” means to obtain only the desired enantiomer, regioisomer or diastereomer by taking advantage of the unique physical properties of said enantiomer, regioisomer or diastereomer (e.g., solubility in a particular solvent or solvent system). “Selectively isolating” a desired enantiomer, regioisomer or diastereomer typically further involves mechanical means (i.e. filtration) to separate the desired enantiomer, regioisomer or diastereomer from the undesired enantiomer, regioisomer or diastereomer (or other impurities).

As used herein, the term “intermediate” refers to a compound or chemical entity in a chemical process that is prepared in a step after the starting material is provided and before the final product is prepared. In some instances, an intermediate is not isolated during the chemical process and is converted to a subsequent compound in situ. For example, a compound may be subjected to successive chemical reactions in just one reactor.

In the above recitations, the term “alkyl”, used either alone or in compound words such as “haloalkyl” includes straight-chain or branched alkyl, such as methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. The term “C₁-C₆ alkanol” alternatively means C₁-C₆ hydroxyalkyl. “Alkoxy” includes, for example, methoxy, ethoxy, n-propyloxy, isopropyloxy and the different butoxy, pentoxy and hexyloxy isomers.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl” or “haloalkenyl”, or when used in descriptions such as “alkyl substituted with halogen” said alkyl may be partially or fully substituted with halogen atoms which may be the same or different. Examples of “haloalkyl” or “alkyl substituted with halogen” include F₃C, ClCH₂, CF₃CH₂ and CF₃CCl₂. The terms “haloalkoxy”, and the like, is defined analogously to the term “haloalkyl”. Examples of “haloalkoxy” include CF₃O—, CCl₃CH₂O—, HCF₂CH₂CH₂O— and CF₃CH₂O—. “Cyano” denotes a —C≡N group. “Nitro” means an NO₂ group.

As used herein, “alkali metal” refers to elements of group 1 of the periodic table, including lithium, sodium, potassium and cesium, preferably sodium or potassium, or cations thereof, such as when used in combination with an anionic counterion to define a chemical compound.

The term “quaternary ammonium bromide salt” refers to a bromide salt of a quaternary ammonium cation having the structure (R⁷)₄N⁺Br⁻, wherein
each R⁷ is independently C₁-C₂₀ alkyl or C₁-C₆ haloalkyl; or phenyl or benzyl, each optionally substituted with up to two R²; or
two adjacent R⁷ substituents are taken together with the nitrogen atom to which they are attached to form a 5 to 8-membered cyclic structure.
   Examples of quaternary ammonium bromide salts include tetrabutylammonium bromide, N-cetyl-N,N,N-trimethylammonium bromide and benzyltriethylammonium bromide.

The total number of carbon atoms in a substituent group is indicated by the “Cᵢ-Cⱼ” prefix where i and j are numbers from 1 to 6. When a compound is substituted with a substituent bearing a subscript that indicates the number of said substituents can exceed 1, said substituents (when they exceed 1) are independently selected from the group of defined substituents, (e.g., (R¹)ₘ, m is 0, 1, 2 or 3). When a group contains a substituent that can be hydrogen, for example (when m=0), then when this substituent is taken as hydrogen, it is recognized that this is equivalent to said group being unsubstituted. When a variable group is shown to be optionally attached to a position, (for example (R¹)ₘ attached to a phenyl group wherein m may be 0), then hydrogen may be at the position even if not recited in the variable group definition. When one or more positions on a group are said to be “not substituted” or “unsubstituted”, then hydrogen atoms are attached to take up any free valency.

As used herein, “adjacent” means that two substituents are near each other but are not directly connected. For example, the term “adjacent R¹ substituents” indicates R¹ substituents that are attached to contiguous carbon atoms, such as in a phenyl group. “Adjacent R⁷ substituents” are geminally attached to a single nitrogen atom.

The term “optionally” when used herein means that the optional condition may or may not be present. For example, when a reaction is conducted optionally in the presence of a solvent, the solvent may or may not be present.

The term “optionally substituted” refers to groups which are unsubstituted or have at least one non-hydrogen substituent that does not extinguish the chemical or biological activity possessed by the unsubstituted analog. As used herein, the following definitions shall apply unless otherwise indicated. The term “optionally substituted with” is used interchangeably with the phrase “unsubstituted or substituted with” or with the term “(un)substituted with”. Unless otherwise indicated, an optionally substituted group may have a substituent at each substitutable position of the group, and each substitution is independent of the other.

This invention comprises racemic mixtures, for example, essentially equal amounts of the enantiomers of 2-bromobutanoic acid. In addition, this invention includes compounds that are enantiomerically enriched compared to the racemic mixture; for example in an enantiomer of compound S-1 or any intermediate in a process described herein for preparing compound S-1. Also included are the essentially pure enantiomers of compound S-1 or any intermediate in a process described herein for preparing compound S-1.

When enantiomerically enriched, one enantiomer is present in greater amounts than the other, and the extent of enrichment can be defined by an expression of enantiomeric excess (“ee”), which is defined as (Fₘₐⱼ−Fₘᵢₙ) 100%, where Fₘₐⱼ is the mole fraction of the dominant enantiomer in the mixture and Fₘᵢₙ is the mole fraction of the lesser enantiomer in the mixture (e.g., an ee of 20% corresponds to a 60:40 ratio of enantiomers).

As used herein, compounds having at least an 80% enantiomeric excess; preferably at least a 90% enantiomeric excess; more preferably at least a 94% enantiomeric excess, at least a 96% enantiomeric excess; at least a 98% enantiomeric excess of a specific isomer are designated as R- or S-, depending on the predominant configuration at the asymmetric center. Of note are essentially enantiomerically pure embodiments (>99% ee) of the more predominant enantiomer. As used herein, compounds having less than 80% enantiomeric excess are designated as scalemic.

Molecular depictions drawn herein generally follow standard conventions for depicting stereochemistry. To indicate stereoconfiguration, bonds rising from the plane of the drawing and towards the viewer are denoted by solid wedges where the broad end of the wedge is attached to the atom rising from the plane of the drawing towards the viewer as shown below, where group B is rising from above the plane of the drawing. Except where specifically indicated, hydrogen atoms attached to the asymmetric center are generally not shown.

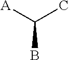

Bonds going below the plane of the drawing and away from the viewer are denoted by dashed wedges where the broad end of the wedge is attached to the atom further away from the viewer, i.e. group B′ is below the plane of the drawing.

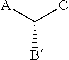

Constant width lines indicate bonds with a direction opposite or neutral relative to bonds shown with solid or dashed wedges; constant width lines also depict bonds in molecules or parts of molecules in which no stereoconfiguration is intended to be specified. Notably as used herein, a constant width line attached to an asymmetric center also represents a condition where the amounts of R- and S-configuration at that center are equal; e.g., a compound with a single asymmetric center is racemic. When a racemic mixture is intended for any specific compound herein, it is denoted with the prefix “rac-”

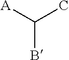

### Racemic Mixture or “Rac”

Wavy lines indicate bonds in molecules or parts of molecules in which no particular stereoconfiguration is intended to be specified. Accordingly, as used herein, a wavy line attached to an asymmetric center represents a condition where the amounts of R- and S-configuration at that center are non-equal but not of sufficiently high enantiomeric excess for either R- or S-configuration; e.g., a compound with a single asymmetric center is scalemic as defined herein. When a scalemic mixture is intended for any specific compound herein, it is denoted with the prefix “scal-”

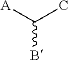

### Scalemic Mixture or “Scal-”

Embodiments of the invention include the following.

Embodiment A1. The method of Embodiment A wherein m is 0, 1 or 2.

Embodiment A2. The method of Embodiment A1 wherein m is 1 or 2.

Embodiment A3. The method of any of Embodiment A, Embodiment A1 or Embodiment A2 wherein
each R¹ is independently halogen, nitro, C₁-C₄ alkyl, C₁-C₄ haloalkyl or phenyl; or
two adjacent R¹ substituents are taken together with the phenyl to which they are attached to form a naphthalenyl ring optionally substituted with up to two R³.

Embodiment A4. The method of Embodiment A3 wherein each R¹ is independently halogen, nitro, C₁-C₄ alkyl or C₁-C₄ haloalkyl.

Embodiment A5. The method of Embodiment A4 wherein each R¹ is independently halogen or C₁-C₄ alkyl.

Embodiment A6. The method of Embodiment A3 wherein m is 2 and two adjacent R¹ substituents are taken together with the phenyl to which they are attached to form an unsubstituted naphthalenyl ring.

Embodiment A7. The method of any of Embodiments A through A6 wherein n is 0, 1 or 2.

Embodiment A8. The method of Embodiment A7 wherein n is 1 or 2.

Embodiment A9. The method of Embodiment A8 wherein each R⁴ is independently halogen, nitro, C₁-C₄ alkyl or C₁-C₄ haloalkyl.

Embodiment A10. The method of Embodiment A9 wherein each R⁴ is independently halogen or C₁-C₄ alkyl.

Embodiment A11. The method of Embodiment A7 wherein n is 0.

Embodiment A12. The method of any of Embodiments A through A11 wherein the compound of Formula 3 is selected from the group consisting of
(αR)-α-methyl-N-(phenylmethyl)-benzenemethanamine,
N-[(1R)-1-phenylethyl]-1-naphthalenemethanamine,
2,4-dichloro-N-[(1R)-1-phenylethyl]-benzenemethanamine,
3,4-dichloro-N-[(1R)-1-phenylethyl]-benzenemethanamine,
2,6-dichloro-N-[(1R)-1-phenylethyl]-benzenemethanamine,
2,4,6-trimethyl-N-[(1R)-1-phenylethyl]-benzenemethanamine,
4-nitro-N-[(1R)-1-phenylethyl]-benzenemethanamine, and
2-methyl-3-phenyl-N-[(1R)-1-phenylethyl]-benzenemethanamine.

Embodiment A13. The method of any of Embodiments A through A12 wherein m is 2 and two adjacent R¹ substituents are taken together with the phenyl to which they are attached to form an unsubstituted 1-naphthalenyl ring; and n is 0; i.e. the compound of Formula 3 is compound 3A [N-[(1R)-1-phenylethyl]-1-naphthalenemethanamine].

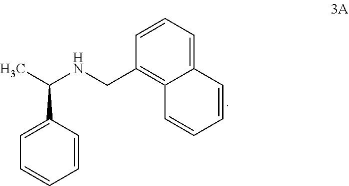 [SMILES: C[C@@H](NCc1cccc2ccccc12)c1ccccc1]

Embodiment A14. The method of any of Embodiments A through A13 wherein compound R-2 is converted to compound S-1 by the method comprising
treating compound R-2 with a C₁-C₆ alkanol to prepare the compound of Formula R-6;

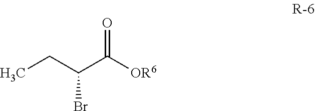

wherein R⁶ is C₁-C₆ alkyl;
treating the compound of Formula R-6 with compound 7

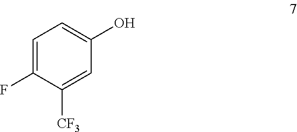 [SMILES: Oc1ccc(F)c(C(F)(F)F)c1]

to prepare the compound of Formula S-8

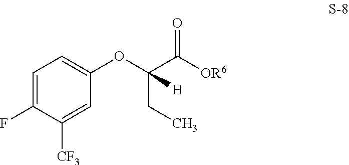

wherein R⁶ is C₁-C₆ alkyl;
and treating the compound of Formula S-8 with compound 9

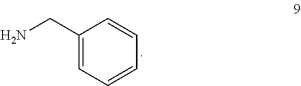

Embodiment A15. The method of Embodiment A14 wherein treating compound R-2 to prepare the compound of Formula R-6 comprises
treating compound R-2 with a chlorinating agent to prepare compound R-10

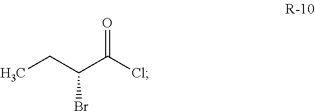

and
treating compound R-10 with a C₁-C₆ alkanol or a salt thereof.

Embodiment A16. The method of Embodiment A15 wherein the chlorinating agent is thionyl chloride.

Embodiment A17. The method of any of Embodiments A14 through A16 wherein R⁶ is CH₃.

Embodiment A18. The method of any of Embodiments A through A13 wherein compound R-2 is converted to compound S-1 by the method comprising
treating compound R-2 with a chlorinating agent to prepare compound R-10

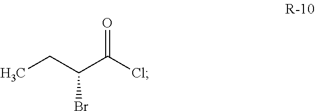

treating compound R-10 with compound 9

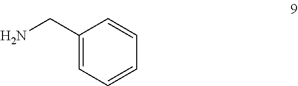

to prepare compound R-11

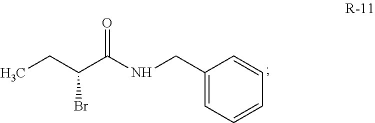

and
treating compound R-11 with compound 7

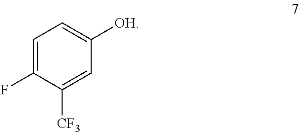 [SMILES: Oc1ccc(F)c(C(F)(F)F)c1]

Embodiment A19. The method of Embodiment A18 wherein the chlorinating agent is thionyl chloride.

Embodiment B1. The method of Embodiment B wherein m is 0, 1 or 2.

Embodiment B2. The method of Embodiment B1 wherein m is 1 or 2.

Embodiment B3. The method of any of Embodiment B, Embodiment B1 or Embodiment B2 wherein
each R¹ is independently halogen, nitro, C₁-C₄ alkyl, C₁-C₄ haloalkyl or phenyl; or
two adjacent R¹ substituents are taken together with the phenyl to which they are attached to form a naphthalenyl ring optionally substituted with up to two R³.

Embodiment B4. The method of Embodiment B3 wherein each R¹ is independently halogen, nitro, C₁-C₄ alkyl or C₁-C₄ haloalkyl.

Embodiment B5. The method of Embodiment B4 wherein each R¹ is independently halogen or C₁-C₄ alkyl.

Embodiment B6. The method of Embodiment B3 wherein m is 2 and two adjacent R¹ substituents are taken together with the phenyl to which they are attached to form an unsubstituted naphthalenyl ring.

Embodiment B7. The method of any of Embodiments B through B6 wherein n is 0, 1 or 2.

Embodiment B8. The method of Embodiment B7 wherein n is 1 or 2.

Embodiment B9. The method of Embodiment B8 wherein each R⁴ is independently halogen, nitro, C₁-C₄ alkyl or C₁-C₄ haloalkyl.

Embodiment B10. The method of Embodiment B9 wherein each R⁴ is independently halogen or C₁-C₄ alkyl.

Embodiment B11. The method of Embodiment B7 wherein n is 0.

Embodiment B12. The method of any of Embodiments B through B11 wherein the compound of Formula 3 is selected from the group consisting of
(αR)-α-methyl-N-(phenylmethyl)-benzenemethanamine,
N-[(1R)-1-phenylethyl]-1-naphthalenemethanamine,
2,4-dichloro-N-[(1R)-1-phenylethyl]-benzenemethanamine,
3,4-dichloro-N-[(1R)-1-phenylethyl]-benzenemethanamine,
2,6-dichloro-N-[(1R)-1-phenylethyl]-benzenemethanamine,
2,4,6-trimethyl-N-[(1R)-1-phenylethyl]-benzenemethanamine,
4-nitro-N-[(1R)-1-phenylethyl]-benzenemethanamine, and
2-methyl-3-phenyl-N-[(1R)-1-phenylethyl]-benzenemethanamine.

Embodiment B13. The method of any of Embodiments B through B12 wherein m is 2 and two adjacent R¹ substituents are taken together with the phenyl to which they are attached to form an unsubstituted 1-naphthalenyl ring; and n is 0; i.e. the compound of Formula 3 is compound 3A [N-[(1R)-1-phenylethyl]-1-naphthalenemethanamine]

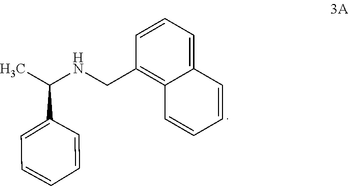 [SMILES: C[C@@H](NCc1cccc2ccccc12)c1ccccc1]

Embodiment B14. The method of any of Embodiments B through B13 wherein compound R-2 is converted to compound S-1 by the method comprising
treating compound R-2 to prepare the compound of Formula R-6;

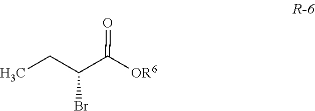

wherein R⁶ is C₁-C₆ alkyl;
treating the compound of Formula R-6 with compound 7

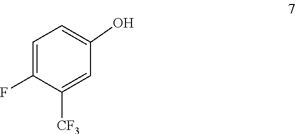 [SMILES: Oc1ccc(F)c(C(F)(F)F)c1]

to prepare the compound of Formula S-8

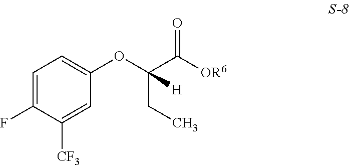

wherein R⁶ is C₁-C₆ alkyl; and
treating the compound of Formula S-8 with compound 9

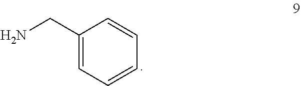

Embodiment B15. The method of Embodiment B14 wherein treating compound R-2 to prepare the compound of Formula R-6 comprises
treating compound R-2 with a chlorinating agent to prepare compound R-10

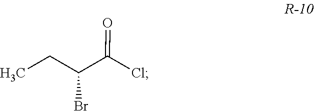

and
treating compound R-10 with a C₁-C₆ alkanol or a salt thereof.

Embodiment B16. The method of Embodiment B15 wherein the chlorinating agent is thionyl chloride.

Embodiment B17. The method of any of Embodiments B14 through B16 wherein R⁶ is CH₃.

Embodiment B18. The method any of Embodiments B through B13 wherein converting compound R-2 to compound S-1 comprises
treating compound R-2 with a chlorinating agent to prepare compound R-10

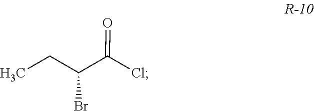

treating compound R-10 with compound 9

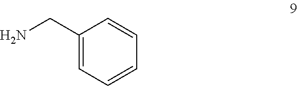

to prepare compound R-11

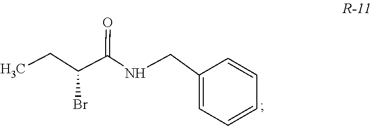

and
treating compound R-11 with compound 7

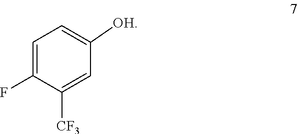 [SMILES: Oc1ccc(F)c(C(F)(F)F)c1]

Embodiment B19. The method of Embodiment B18 wherein the chlorinating agent is thionyl chloride.

Embodiment C1. The method of Embodiment C wherein m is 0, 1 or 2.

Embodiment C2. The method of Embodiment C1 wherein m is 1 or 2.

Embodiment C3. The method of any of Embodiment C, Embodiment C1 or Embodiment C2 wherein
each R¹ is independently halogen, nitro, C₁-C₄ alkyl, C₁-C₄ haloalkyl or phenyl; or
two adjacent R¹ substituents are taken together with the phenyl to which they are attached to form a naphthalenyl ring optionally substituted with up to two R³.

Embodiment C4. The method of Embodiment C3 wherein each R¹ is independently halogen, nitro, C₁-C₄ alkyl or C₁-C₄ haloalkyl.

Embodiment C5. The method of Embodiment C4 wherein each R¹ is independently halogen or C₁-C₄ alkyl.

Embodiment C6. The method of Embodiment C3 wherein m is 2 and two adjacent R¹ substituents are taken together with the phenyl to which they are attached to form an unsubstituted naphthalenyl ring.

Embodiment C7. The method of any of Embodiments C through C6 wherein n is 0, 1 or 2.

Embodiment C8. The method of Embodiment C7 wherein n is 1 or 2.

Embodiment C9. The method of Embodiment C8 wherein each R⁴ is independently halogen, nitro, C₁-C₄ alkyl or C₁-C₄ haloalkyl.

Embodiment C10. The method of Embodiment C9 wherein each R⁴ is independently halogen or C₁-C₄ alkyl.

Embodiment C11. The method of Embodiment C7 wherein n is 0.

Embodiment C12. The method of any of Embodiments C through C11 wherein the compound of Formula 3 is selected from the group consisting of
(αR)-α-methyl-N-(phenylmethyl)-benzenemethanamine,
N-[(1R)-1-phenylethyl]-1-naphthalenemethanamine,
2,4-dichloro-N-[(1R)-1-phenylethyl]-benzenemethanamine,
3,4-dichloro-N-[(1R)-1-phenylethyl]-benzenemethanamine,
2,6-dichloro-N-[(1R)-1-phenylethyl]-benzenemethanamine,
2,4,6-trimethyl-N-[(1R)-1-phenylethyl]-benzenemethanamine,
4-nitro-N-[(1R)-1-phenylethyl]-benzenemethanamine, and
2-methyl-3-phenyl-N-[(1R)-1-phenylethyl]-benzenemethanamine.

Embodiment C13. The method of any of Embodiments C through C12 wherein m is 2 and two adjacent R¹ substituents are taken together with the phenyl to which they are attached to form an unsubstituted 1-naphthalenyl ring; and n is 0; i.e. the compound of Formula 3 is compound 3A [N-[(1R)-1-phenylethyl]-1-naphthalenemethanamine]

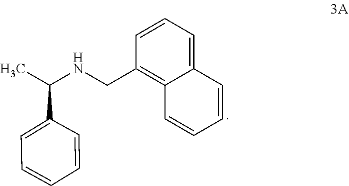 [SMILES: C[C@@H](NCc1cccc2ccccc12)c1ccccc1]

Embodiment C14. The method of any of Embodiments C through C13 wherein compound R-2 is converted to compound S-1 by the method comprising treating compound R-2 to prepare the compound of Formula R-6;

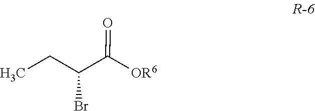

wherein R⁶ is C₁-C₆ alkyl;
treating the compound of Formula R-6 with compound 7

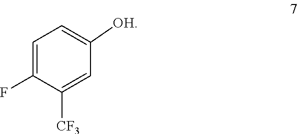 [SMILES: Oc1ccc(F)c(C(F)(F)F)c1]

to prepare the compound of Formula S-8

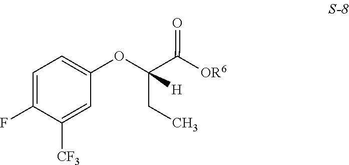

wherein R⁶ is C₁-C₆ alkyl; and
treating the compound of Formula S-8 with compound 9

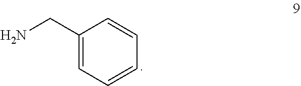

Embodiment C15. The method of Embodiment C14 wherein treating compound R-2 to prepare the compound of Formula R-6 comprises
treating compound R-2 with a chlorinating agent to prepare compound R-10

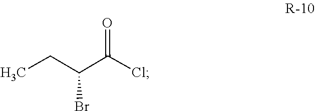

treating compound R-10 with a C₁-C₆ alkanol or a salt thereof.

Embodiment C16. The method of Embodiment C15 wherein the chlorinating agent is thionyl chloride.

Embodiment C17. The method of any of Embodiments C₁₄ through C₁₆ wherein R⁶ is CH₃.

Embodiment C18. The method any of Embodiments C through C13 wherein converting compound R-2 to compound S-1 comprises
treating compound R-2 with a chlorinating agent to prepare a compound of Formula R-10;
treating compound R-10 with compound 9 to prepare compound R-11

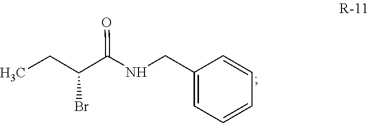

and
treating compound R-11 with compound 7.

Embodiment C19. The method of Embodiment C18 wherein the chlorinating agent is thionyl chloride.

Embodiment D1. The method of Embodiment D wherein m is 0, 1 or 2.

Embodiment D2. The method of Embodiment D1 wherein m is 1 or 2.

Embodiment D3. The method of any of Embodiment D, Embodiment D1 or Embodiment D2 wherein
each R¹ is independently halogen, nitro, C₁-C₄ alkyl, C₁-C₄ haloalkyl or phenyl; or
two adjacent R¹ substituents are taken together with the phenyl to which they are attached to form a naphthalenyl ring optionally substituted with up to two R³.

Embodiment D4. The method of Embodiment D3 wherein each R¹ is independently halogen, nitro, C₁-C₄ alkyl or C₁-C₄ haloalkyl.

Embodiment D5. The method of Embodiment D4 wherein each R¹ is independently halogen or C₁-C₄ alkyl.

Embodiment D6. The method of Embodiment D3 wherein m is 2 and two adjacent R¹ substituents are taken together with the phenyl to which they are attached to form an unsubstituted naphthalenyl ring.

Embodiment D7. The method of any of Embodiments D through D6 wherein n is 0, 1 or 2.

Embodiment D8. The method of Embodiment D7 wherein n is 1 or 2.

Embodiment D9. The method of Embodiment D8 wherein each R⁴ is independently halogen, nitro, C₁-C₄ alkyl or C₁-C₄ haloalkyl.

Embodiment D10. The method of Embodiment D9 wherein each R⁴ is independently halogen or C₁-C₄ alkyl.

Embodiment D11. The method of Embodiment D7 wherein n is 0.

Embodiment D12. The method of any of Embodiments D through D11 wherein the compound of Formula 3 is selected from the group consisting of
(αR)-α-methyl-N-(phenylmethyl)-benzenemethanamine,
N-[(1R)-1-phenylethyl]-1-naphthalenemethanamine,
2,4-dichloro-N-[(1R)-1-phenylethyl]-benzenemethanamine,
3,4-dichloro-N-[(1R)-1-phenylethyl]-benzenemethanamine,
2,6-dichloro-N-[(1R)-1-phenylethyl]-benzenemethanamine,
2,4,6-trimethyl-N-[(1R)-1-phenylethyl]-benzenemethanamine,
4-nitro-N-[(1R)-1-phenylethyl]-benzenemethanamine, and
2-methyl-3-phenyl-N-[(1R)-1-phenylethyl]-benzenemethanamine.

Embodiment D13. The method of any of Embodiments D through D12 wherein m is 2 and two adjacent R¹ substituents are taken together with the phenyl to which they are attached to form an unsubstituted 1-naphthalenyl ring; and n is 0; i.e. the compound of Formula 3 is compound 3A [N-[(1R)-1-phenylethyl]-1-naphthalenemethanamine]

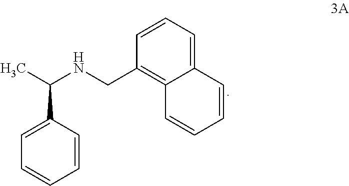 [SMILES: C[C@@H](NCc1cccc2ccccc12)c1ccccc1]

Embodiment D14. The method of any of Embodiments D through D13 wherein the chlorinating agent is thionyl chloride.

Embodiment E1. The method of Embodiment E wherein m is 0, 1 or 2.

Embodiment E2. The method of Embodiment E1 wherein m is 1 or 2.

Embodiment E3. The method of any of Embodiment E, Embodiment E1 or Embodiment E2 wherein
each R¹ is independently halogen, nitro, C₁-C₄ alkyl, C₁-C₄ haloalkyl or phenyl; or
two adjacent R¹ substituents are taken together with the phenyl to which they are attached to form a naphthalenyl ring optionally substituted with up to two R³.

Embodiment E4. The method of Embodiment E3 wherein each R¹ is independently halogen, nitro, C₁-C₄ alkyl or C₁-C₄ haloalkyl.

Embodiment E5. The method of Embodiment E4 wherein each R¹ is independently halogen or C₁-C₄ alkyl.

Embodiment E6. The method of Embodiment E3 wherein m is 2 and two adjacent R¹ substituents are taken together with the phenyl to which they are attached to form an unsubstituted naphthalenyl ring.

Embodiment E7. The method of any of Embodiments E through E6 wherein n is 0, 1 or 2.

Embodiment E8. The method of Embodiment E7 wherein n is 1 or 2.

Embodiment E9. The method of Embodiment E8 wherein each R⁴ is independently halogen, nitro, C₁-C₄ alkyl or C₁-C₄ haloalkyl.

Embodiment E10. The method of Embodiment E9 wherein each R⁴ is independently halogen or C₁-C₄ alkyl.

Embodiment E11. The method of Embodiment E7 wherein n is 0.

Embodiment E12. The method of any of Embodiments E through E11 wherein the compound of Formula 3 is selected from the group consisting of
(αR)-α-methyl-N-(phenylmethyl)-benzenemethanamine,
N-[(1R)-1-phenylethyl]-1-naphthalenemethanamine,
2,4-dichloro-N-[(1R)-1-phenylethyl]-benzenemethanamine,
3,4-dichloro-N-[(1R)-1-phenylethyl]-benzenemethanamine,
2,6-dichloro-N-[(1R)-1-phenylethyl]-benzenemethanamine,
2,4,6-trimethyl-N-[(1R)-1-phenylethyl]-benzenemethanamine,
4-nitro-N-[(1R)-1-phenylethyl]-benzenemethanamine, and
2-methyl-3-phenyl-N-[(1R)-1-phenylethyl]-benzenemethanamine.

Embodiment E13. The method of Embodiment E6 wherein m is 2 and two adjacent R¹ substituents are taken together with the phenyl to which they are attached to form an unsubstituted 1-naphthalenyl ring; and n is 0; i.e. the compound of Formula 3 is compound 3A [N-[(1R)-1-phenylethyl]-1-naphthalenemethanamine]

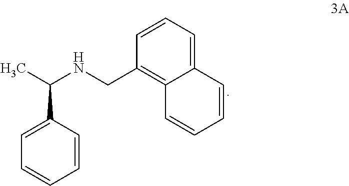 [SMILES: C[C@@H](NCc1cccc2ccccc12)c1ccccc1]

Embodiment F1. The method of Embodiment F wherein compound scal-2 is predominantly (S)-2-bromobutanoic acid.

Embodiment F2. The method of Embodiment F or Embodiment F1 wherein compound scal-2 is treated with hydrobromic acid.

Embodiment F3. The method of Embodiment F or Embodiment F1 wherein compound scal-2 is treated with a quaternary ammonium bromide salt.

Embodiment F4. The method of Embodiment F3 wherein the quaternary ammonium bromide salt is tetrabutylammonium bromide.

Embodiment G1. The salt of Embodiment G wherein m is 0, 1 or 2.

Embodiment G2. The salt of Embodiment G1 wherein m is 1 or 2.

Embodiment G3. The salt of Embodiment G, Embodiment G1 or Embodiment G2 wherein
each R¹ is independently halogen, nitro, C₁-C₄ alkyl, C₁-C₄ haloalkyl or phenyl; or
two adjacent R¹ substituents are taken together with the phenyl to which they are attached to form a naphthalenyl ring optionally substituted with up to two R³.

Embodiment G4. The salt of Embodiment G3 wherein each R¹ is independently halogen, nitro, C₁-C₄ alkyl or C₁-C₄ haloalkyl.

Embodiment G5. The salt of Embodiment G4 wherein each R¹ is independently halogen or C₁-C₄ alkyl.

Embodiment G6. The salt of Embodiment G3 wherein m is 2 and two adjacent R¹ substituents are taken together with the phenyl to which they are attached to form an unsubstituted naphthalenyl ring.

Embodiment G7. The salt of any of Embodiments G through G6 wherein n is 0, 1 or 2.

Embodiment G8. The salt of Embodiment G7 wherein n is 1 or 2.

Embodiment G9. The salt of Embodiment G8 wherein each R⁴ is independently halogen, nitro, C₁-C₄ alkyl or C₁-C₄ haloalkyl.

Embodiment G10. The salt of Embodiment G9 wherein each R⁴ is independently halogen or C₁-C₄ alkyl.

Embodiment G11. The salt of Embodiment G7 wherein n is 0.

Embodiment G12. The salt of any of Embodiments G through G11 wherein the salt of Formula 4 comprises an amine selected from the group consisting of
(αR)-α-methyl-N-(phenylmethyl)-benzenemethanamine,
N-[(1R)-1-phenylethyl]-1-naphthalenemethanamine,
2,4-dichloro-N-[(1R)-1-phenylethyl]-benzenemethanamine,
3,4-dichloro-N-[(1R)-1-phenylethyl]-benzenemethanamine,
2,6-dichloro-N-[(1R)-1-phenylethyl]-benzenemethanamine,
2,4,6-trimethyl-N-[(1R)-1-phenylethyl]-benzenemethanamine,
4-nitro-N-[(1R)-1-phenylethyl]-benzenemethanamine, and
2-methyl-3-phenyl-N-[(1R)-1-phenylethyl]-benzenemethanamine.

Embodiment G13. The salt of Embodiment G6 wherein m is 2 and two adjacent R¹ substituents are taken together with the phenyl to which they are attached to form an unsubstituted 1-naphthalenyl ring; and n is 0, i.e. the salt of Formula 4A

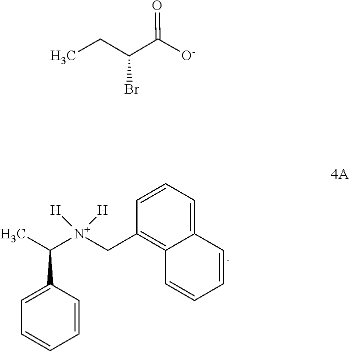

Embodiments of this invention, including Embodiments A through A19, B through B19, C through C19, D through D14, E through E13, F through F4 and G through G13 above as well as any other embodiments (including Embodiments P1 through P10) described herein, can be combined in any manner, and the descriptions of variables in the embodiments pertain not only to compounds S-1 but also to the starting compounds and intermediate compounds of Formulae 2 through 11, useful for preparing compound S-1.

Preferred Embodiments include the following.

Embodiment P1. The method of any of Embodiments A, B, C, D or E above wherein m is 1 or 2;
n is 0; and
each R¹ is independently halogen, nitro, C₁-C₄ alkyl, C₁-C₄ haloalkyl or phenyl; or
two adjacent R¹ substituents are taken together with the phenyl to which they are attached to form an unsubstituted naphthalenyl ring.

Embodiment P2. The method of any of Embodiments A, B, C, D or E above wherein the compound of Formula 3 is selected from the group consisting of
(αR)-α-methyl-N-(phenylmethyl)-benzenemethanamine,
N-[(1R)-1-phenylethyl]-1-naphthalenemethanamine,
2,4-dichloro-N-[(1R)-1-phenylethyl]-benzenemethanamine,
3,4-dichloro-N-[(1R)-1-phenylethyl]-benzenemethanamine,
2,6-dichloro-N-[(1R)-1-phenylethyl]-benzenemethanamine,
2,4,6-trimethyl-N-[(1R)-1-phenylethyl]-benzenemethanamine,
4-nitro-N-[(1R)-1-phenylethyl]-benzenemethanamine, and
2-methyl-3-phenyl-N-f[(1R)-1-phenylethyl]-benzenemethanamine.

Embodiment P3. The method of any of Embodiments A, B, C, D or E above wherein m is 2 and two adjacent R¹ substituents are taken together with the phenyl to which they are attached to form an unsubstituted 1-naphthalenyl ring; and n is 0.

Embodiment P4. The method of any of Embodiments A, B, C, D or E above wherein compound R-2 is converted to the compound of Formula S-8

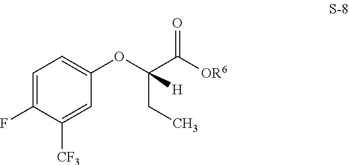

wherein R⁶ is C₁-C₆ alkyl; and
the compound of Formula S-8 is treated with compound 9

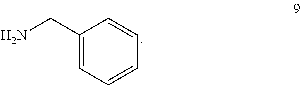

Embodiment P5. The method of any of Embodiments A, B or C above wherein compound R-2 is converted to a compound of Formula S-8 by the method comprising treating compound R-2 to prepare a compound of Formula R-6

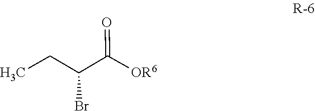

wherein R⁶ is C₁-C₆ alkyl; and
the compound of Formula R-6 is treated with compound 7

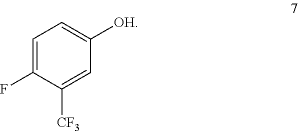 [SMILES: Oc1ccc(F)c(C(F)(F)F)c1]

Embodiment P6. The method of any of Embodiments A, B, C or D above wherein compound R-2 is treated with a chlorinating agent to prepare compound R-10

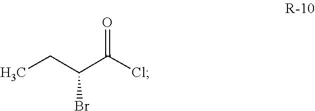

compound R-10 is treated with compound 9

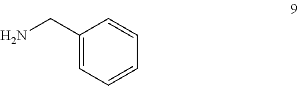

to prepare compound R-11

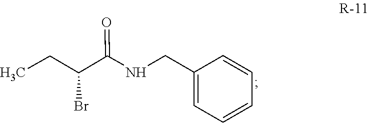

and
compound R-11 is treated with compound 7

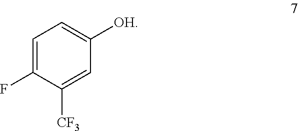 [SMILES: Oc1ccc(F)c(C(F)(F)F)c1]

Embodiment P7. The method of Embodiment P6 wherein the chlorinating agent is thionyl chloride.

Embodiment P8. The salt of Embodiment G wherein
m is 1 or 2;
n is 0; and
each R¹ is independently halogen, nitro, C₁-C₄ alkyl, C₁-C₄ haloalkyl or phenyl; or
two adjacent R¹ substituents are taken together with the phenyl to which they are attached to form an unsubstituted naphthalenyl ring.

Embodiment P9. The salt of Embodiment P7 comprising a salt of an amine selected from the group consisting of
(αR)-α-methyl-N-(phenylmethyl)-benzenemethanamine,
N-[(1R)-1-phenylethyl]-1-naphthalenemethanamine,
2,4-dichloro-N-[(1R)-1-phenylethyl]-benzenemethanamine,
3,4-dichloro-N-[(1R)-1-phenylethyl]-benzenemethanamine,
2,6-dichloro-N-[(1R)-1-phenylethyl]-benzenemethanamine,
2,4,6-trimethyl-N-[(1R)-1-phenylethyl]-benzenemethanamine,
4-nitro-N-[(1R)-1-phenylethyl]-benzenemethanamine, and
2-methyl-3-phenyl-N-[(1R)-1-phenylethyl]-benzenemethanamine.

Embodiment P10. The salt of Embodiment P7 wherein m is 2 and two adjacent R¹ substituents are taken together with the phenyl to which they are attached to form an unsubstituted 1-naphthalenyl ring; and n is 0.

In the following Schemes the definitions of R¹, R², R³, R⁴ and m in the compounds of Formulae 3 through 11 below are as defined above in the Summary of the Invention and description of embodiments unless otherwise indicated.

The methods described herein provide and efficient and robust synthesis of compound S-1.

As summarized in Scheme 1, a compound of Formula S-1 can be prepared from compound R-2, wherein compound R-2 is obtained by resolution of compound rac-2, as described in greater detail with reference to Scheme 2. Conversion of compound R-2 to compound S-1 can be accomplished by any of several reaction sequences subsequently described herein.

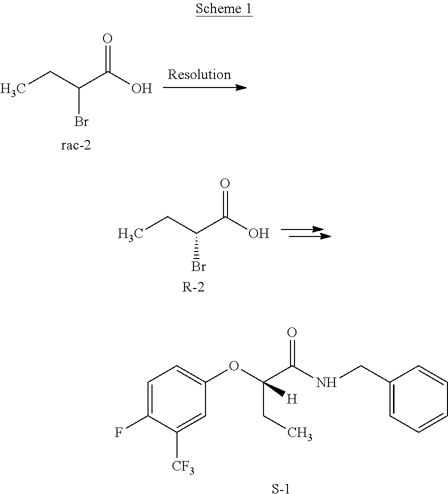

Obtaining acids of high enantiomeric purity can be accomplished in several ways, including catalytic asymmetric synthesis, chromatographic resolution, extraction resolution, membrane resolution, enzymatic resolution and diastereomeric salt resolution. Optical resolution of racemic substrates through diastereomeric salt formation is one of the more practical and economical approaches for industrial-scale production. However, the efficiency of diasteromeric salt resolutions depends on the differential solubility of the diasteromeric salts in at least one solvent. For a given racemate, finding a suitable resolving agent/solvent combination is largely a matter of trial and error, a time-consuming and labor-intensive process. Obtaining a high enantiomeric excess may also require multiple recrystallizations of the diastereomeric salt, which can be very detrimental to industrial processes.

Resolution of 2-haloacids using optically active 1-(1-naphthyl)ethylamine has been disclosed (JPS61227549). Resolution of 4-chloromandelic acid using (R)-(+)-benzyl-1-phenylethylamine has been disclosed (Molecules 2018, 23, 3354).

As shown in Scheme 2, resolution of racemic 2-bromobutanoic acid, compound rac-2, can be achieved with high efficiency by treatment with a compound of Formula 3, having the R-configuration at the asymmetric center. Treatment of rac-2 with a compound of Formula 3 provides the R,R- and R,S-diastereomeric salts of the compound of Formula 3 with either R- or S-2-bromobutanoic acid, respectively. Suitable solvents include ketones such as acetone and methyl isobutyl ketone (MIBK), alcohols, optionally mixed with water, such as methanol, ethanol and isopropanol, polar aprotic solvents such as acetonitrile and ethyl acetate, and hydrocarbons such as hexane, petroleum ether, heptane and toluene, and mixtures thereof. The R,R-diastereomeric salt of Formula 4 is generally the less soluble or more stable salt and can be selectively isolated by filtration.

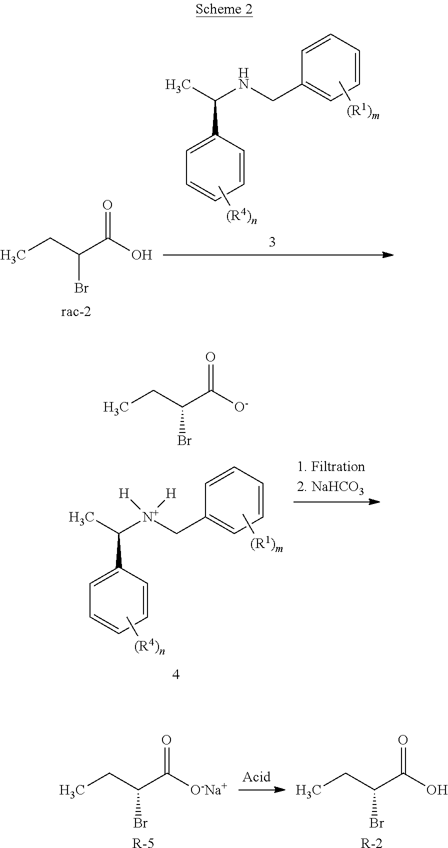

The resulting solid salt of Formula 4 is treated with aqueous base, such as sodium bicarbonate, to provide the water-soluble sodium salt of Formula R-5. Extraction with organic solvents such as toluene can recover the resolving agent of Formula 3 for use in subsequent resolutions. Treatment of compound R-5 with acid provides compound R-2, which can be extracted from the aqueous phase with a suitable organic solvent, such as toluene.

As shown in Scheme 3, compounds of Formula 3 can be prepared by treatment of optionally substituted (R)-1-phenylethylamine (i.e. a compound of Formula 13) with the desired benzyl halide or naphthalenylmethyl halide, typically in the presence of an additional base such as potassium carbonate, and optionally in a suitable solvent. Certain compounds of Formula 3 are disclosed in JP2005023055. Suitable additional bases for the reaction include alkali metal alkoxides such as sodium isopropoxide and potassium tert-butoxide; or alkali metal hydroxides such as potassium hydroxide and sodium hydroxide; or alkali metal carbonates and bicarbonates such as sodium bicarbonate, potassium bicarbonate, sodium carbonate, potassium carbonate and cesium carbonate. A preferred base is potassium carbonate. Suitable solvents include acetonitrile, dichloromethane, dichloroethane, toluene, tetrahydrofuran, dimethyl sulfoxide or N,N-dimethylformamide. Preferred solvents include N,N-dimethylformamide.

Preferred compounds of Formula 3 include those wherein n is 0 and/or each R¹ is independently halogen, nitro, C₁-C₄ alkyl or phenyl; or two adjacent R¹ substituents are taken together with the phenyl to which they are attached to form an unsubstituted naphthalenyl ring.

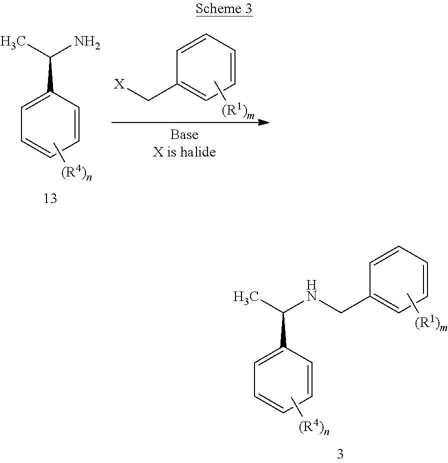

More preferred is compound 3A (See Scheme 4), most preferably when used with a solvent mixture of heptane and MIBK. Using the most preferred combination of compound 3A with a mixture of heptane and MIBK, compound R-2 was obtained in 38% yield (76% of the available R-enantiomer in rac-2) with 96% ee without the need for recrystallization of the compound of Formula 4.

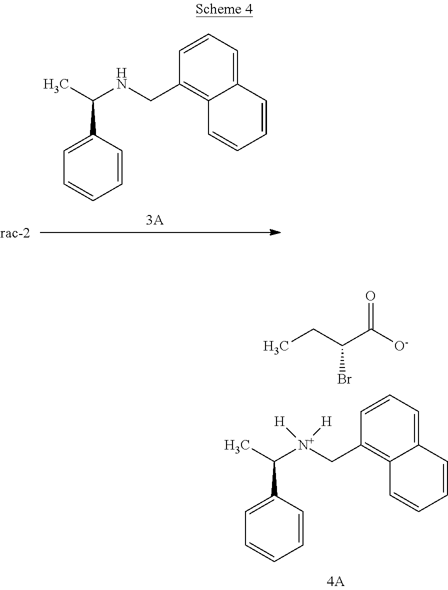

One can appreciate that the procedure summarized in Scheme 2 can be used to obtain compound S-2, if desired, with equal efficiency if the S-enantiomer of a compound of Formula 3 is used.

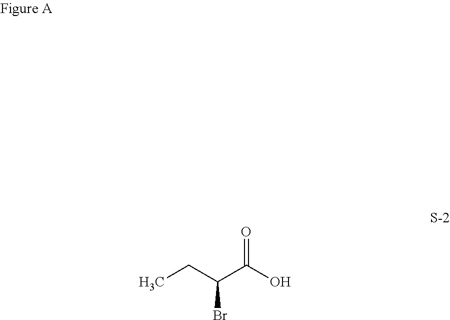

R-2-halobutanoic acids can also be obtained by treatment of racemic 2-halobutanoic acids with 2-haloacid dehalogenase or haloalkane dehalogenases, which selectively react with the S-halo enantiomer, resulting in R-2-halobutanoic acids in high enantiomeric purity (JPH04325096; JPH02238895).

For industrial applicability and avoidance of waste, it is preferred that the undesired enantiomer in the resolution can be recycled to racemic material to be reused to prepare the desired enantiomer. This can be accomplished as summarized in Scheme 5. The mother liquors and washes obtained from the filtration of the solid product R,R-diasteromeric salt of Formula 4 can be treated as described in reference to Scheme 3 to obtain a scalemic mixture of compound scal-2 that is predominantly S-2-bromobutanoic acid with an ee of about 70 to 80%, such as about 74 to 78%. Compound scal-2 can be treated with concentrated hydrobromic acid or a quaternary ammonium bromide salt to provide the compound of rac-2 in essentially 0% ee. A notable quaternary ammonium bromide salt is tetrabutylammonium bromide.

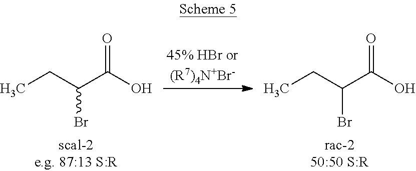

As shown in Scheme 6, compound R-2 can be converted to a compound of Formula R-6 by treatment with a C₁-C₆ alkanol by acid-catalyzed esterification or dehydration with water-absorbing agents such as zeolites. Preferred are the methyl or ethyl ester, and more preferred is the methyl ester. Alternatively, compound R-2 can be converted to the compound of Formula R-6 by treatment with a chlorinating agent to prepare the compound Formula R-10 followed by treatment with a C₁-C₆ alkanol. Suitable chlorinating agents include POCl₃, SOCl₂, (COCl)₂ or COCl₂. Thionyl chloride, SOCl₂, is a preferred chlorinating agent. Suitable solvents include acetonitrile, dichloroethane, toluene, tetrahydrofuran, dimethyl sulfoxide or N,N-dimethylformamide. Preferred solvents include N,N-dimethylformamide, dichloroethane, toluene or acetonitrile, more preferably toluene.

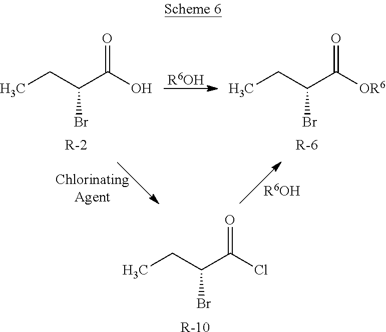

Compounds of Formula R-6 can also be prepared by kinetic resolution of the compound of Formula rac-6 using lipase enzymes (CN105063120).

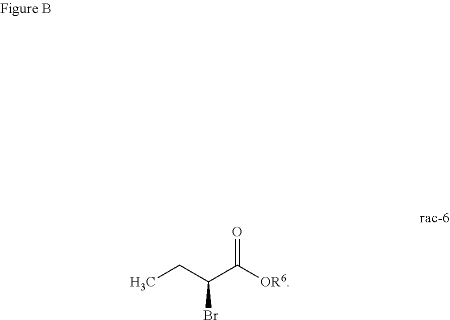

As shown in Scheme 7, the compound of Formula R-6 can be treated with a compound of Formula 7 in the presence of a base to provide the compound of Formula S-8. Suitable solvents include acetonitrile, dichloroethane, toluene, isopropanol, tetrahydrofuran, dimethyl sulfoxide or N,N-dimethylformamide. Preferred solvents include dichloroethane, toluene, acetonitrile or N,N-dimethylformamide, more preferably toluene. Suitable additional bases for the reaction include alkali metal hydrides such as sodium hydride; or alkali metal alkoxides such as sodium isopropoxide and potassium tert-butoxide; or alkali metal hydroxides such as potassium hydroxide and sodium hydroxide; or alkali metal carbonates and bicarbonates such as sodium bicarbonate, potassium bicarbonate, sodium carbonate, potassium carbonate and cesium carbonate; or bases such as lithium bis(trimethylsilyl)amide, sodium bis(trimethylsilyl)amide and lithium diisopropylamide; or tertiary amines such as triethylamine and diisopropylethylamine. Preferred bases include sodium hydroxide, potassium hydroxide, sodium bicarbonate, potassium bicarbonate, sodium carbonate or potassium carbonate, preferably as an aqueous solution.

The compound of Formula S-8 can be treated with compound 9 (i.e. benzyl amine) to provide compound S-1. Preferably, the treatment comprises heating the compound of Formula S-8 with about 2 to 5 molar equivalents of compound 9, such as about three equivalents, at about 100 to 125° C., such as about 110 to 120° C. Optionally, a solvent such as toluene can be used. The crude material obtained after removal of excess benzyl amine can be recrystallized from a mixture of isopropanol and water to provide compound S-1.

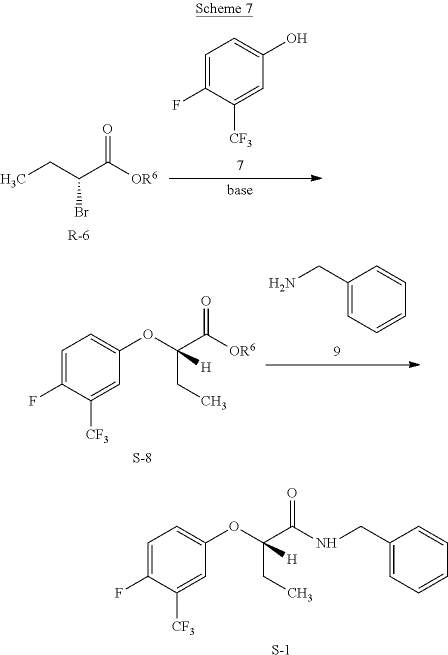

Alternatively, as shown in Scheme 8, compound R-10, prepared as in Scheme 6, can be treated with a compound of Formula 9 in the presence of an additional base to prepare compound R-11. Suitable solvents include acetonitrile, dichloroethane, toluene, tetrahydrofuran, dimethyl sulfoxide or N,N-dimethylformamide. Preferred solvents include N,N-dimethylformamide, dichloroethane, toluene or acetonitrile, more preferably toluene. Suitable additional bases for the reaction include alkali metal hydrides such as sodium hydride; or alkali metal alkoxides such as sodium isopropoxide and potassium tert-butoxide; or alkali metal hydroxides such as potassium hydroxide and sodium hydroxide; or alkali metal carbonates and bicarbonates such as sodium bicarbonate, potassium bicarbonate, sodium carbonate, potassium carbonate and cesium carbonate; or bases such as lithium bis(trimethylsilyl)amide, sodium bis(trimethylsilyl)amide and lithium diisopropylamide; or tertiary amines such as triethylamine and diisopropylethylamine. Preferred bases include sodium hydroxide, potassium hydroxide, sodium bicarbonate, potassium bicarbonate, sodium carbonate or potassium carbonate, preferably as an aqueous solution.

Compound R-11 can be treated with compound 7 in the presence of an additional base to prepare compound S-1. Suitable solvents include acetonitrile, dichloroethane, toluene, isopropanol, tetrahydrofuran, dimethyl sulfoxide or N,N-dimethylformamide. Preferred solvents include N,N-dimethylformamide, dichloroethane, toluene or acetonitrile, more preferably toluene. Suitable additional bases for the reaction include alkali metal hydrides such as sodium hydride; or alkali metal alkoxides such as sodium isopropoxide and potassium tert-butoxide; or alkali metal hydroxides such as potassium hydroxide and sodium hydroxide; or alkali metal carbonates and bicarbonates such as sodium bicarbonate, potassium bicarbonate, sodium carbonate, potassium carbonate and cesium carbonate; or bases such as lithium bis(trimethylsilyl)amide, sodium bis(trimethylsilyl)amide and lithium diisopropylamide; or tertiary amines such as triethylamine and diisopropylethylamine. Preferred bases include sodium hydroxide, potassium hydroxide, sodium bicarbonate, potassium bicarbonate, sodium carbonate or potassium carbonate, preferably as an aqueous solution.

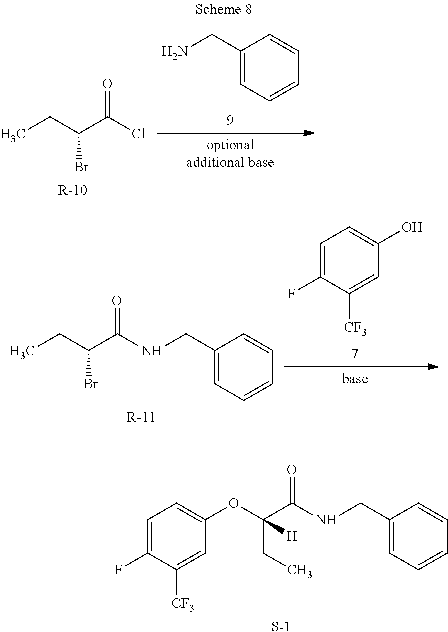

In some embodiments, each of compounds of Formulae R-2, R-6, R-10 and R-11 can be isolated after preparation and before being carried into the next step. Alternatively, two or more of the steps from compound R-2 to compound S-1 can be combined without isolating the intermediate compound. For example, if compound R-2 is extracted from the aqueous phase after acidification with toluene, it can be treated with the chlorinating agent without isolation to prepare compound R-10. In other embodiments, conversion of compound R-2 to the compound of Formula R-6 or compound R-11 can be carried out without isolating compound R-10. In another embodiment, compound R-10 can be converted to compound S-1 without isolating compound R-11. In another embodiment, conversion of compound R-2 to compound S-1 can be accomplished without isolating compounds R-10 and R-11.

Compound R-11 can also be prepared by kinetic resolution of compound rac-11 using haloalkane dehalogenases (Adv. Synth. Cata. 2011, 353, 931-944).

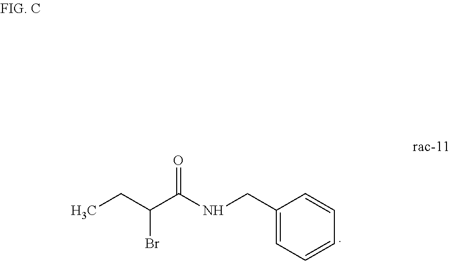

It is recognized that some reagents and reaction conditions described above for preparing compounds of Formulae 1-11 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formulae 1-11. One skilled in the art will also recognize that it may be necessary to perform a combination of the steps illustrated in the above schemes in an order other than that implied by the particular sequence presented to prepare the compounds of Formulae 1-11. One skilled in the art will also recognize that compounds of Formulae 1-11 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. Percentages are by weight. The abbreviation “h” stands for “hour” or “hours”. The abbreviation “GCA” stands for “gas chromatographic area”.

### Synthesis Example 1

### Step 1: Preparation of N-[(1R)-1-phenylethyl]-1-naphthalenemethanamine

A three-liter round bottomed flask fitted with stirrer, condenser and thermometer pocket was charged with N,N-dimethylformamide (1000 g), (R)-1-phenylethanamine (243.10 g, 2 mol) and potassium carbonate (423.10 g, 3.0 mol). To this mixture, 1-(chloromethyl) naphthalene (347 g, 1.959 mol) was added slowly at 28° C. The resulting slurry was heated to 45-46° C. and maintained at that temperature for 13 h. The reaction mass was cooled to 27-28° C. and salts were removed by filtration and washed with N,N-dimethylformamide (2×250 g). The combined N,N-dimethylformamide filtrate was concentrated by distillation under reduced pressure to provide the title compound (535.0 g). Purity by GCA was 95.98%, and yield was 98.25%.

### Synthesis Example 2

### Resolution of Racemic 2-Bromobutanoic Acid

### Step 1: Preparation of the Salt of N-[(1R)-1-phenylethyl]-1-naphthalenemethanamine and (R)-2-bromobutanoic acid

To a three-liter round bottomed flask fitted with stirrer, condenser and thermometer pocket were charged racemic 2-bromobutanoic acid (338.0 g, 2.0 mol), heptane (308 g) and methylisobutyl ketone (252 g). The mixture was heated to about 70° C. To this mixture, a solution of the title compound of Synthesis Example 1 (525.37 g, 2.0 mol) in heptane (132 g) and methylisobutyl ketone (108 g) was added slowly over 1 h at 67-70° C. The resulting slurry was maintained at that temperature for 4 h. The reaction mass was cooled to 28-30° C., maintained at that temperature for 30 minutes and then filtered. The filter cake was washed with methylisobutyl ketone (3×200 g). The crude diastereomeric salt (384.2 g, yield 44.85%) was obtained as a solid. The crude product was taken up in methylisobutyl ketone (500 g) and heated to 50° C. and maintained at that temperature for 1.5 h. The slurry was cooled to 28-30° C. and filtered. The filter cake was washed with 2×200 g of methylisobutyl ketone. The solid diastereomeric salt (364.1 g, yield 42.5%) was obtained.

### Step 2: Preparation of (R)-2-Bromobutanoic Acid

To a two-liter round bottomed flask fitted with stirrer, condenser and thermometer pocket were charged the title compound of Step 1 (362 g, 0.4225 mol), toluene (422.6 g), water (502.0 g) and sodium bicarbonate (90.60 g). The resulting mixture was heated to 38-40° C. and maintained at that temperature for 2 h. The organic layer was separated and the aqueous layer was extracted with 211 g of toluene. The aqueous layer was acidified with 34% HCl (124.0 g, 1.15 mol) at 25° C. Toluene (660 g) was added and the resulting mixture was stirred for 1 h. The organic and aqueous layers were separated and the aqueous layer was extracted with toluene (4×230 g). The combined organic phases were concentrated to dryness to obtained the title compound (128 g) with purity (GCA) of 99.16% and yield of 38% (76% of the available R-isomer), R:S 98:2, ee 96%.

### Synthesis Example 3

### Step 1: Racemization of Scalemic 2-Bromobutanoic Acid

The combined mother liquors and washings obtained from the filtration of the solid product of Step 1, Synthesis Example 2 were treated according to the procedure of Step 2, Synthesis Example 2 to recover 170.43 g of a scalemic mixture of 87% (S)-2-bromobutanoic acid and 13% (R)-2-bromobutanoic acid (74% ee).

To a three-liter round bottomed flask fitted with stirrer, condenser and thermometer pocket were charged water (178.56 g), the scalemic mixture of 2-bromobutanoic acid obtained above (170.43 g, 1 mol) and 45% HBr solution (17.98 g, 0.1 mol). The resulting clear solution was heated to about 78-80° C. and maintained at that temperature for about 6 h. The reaction mixture was cooled to 27-30° C. and extracted thrice with heptane (1×340 g and 2×170 g). The combined organic phases were concentrated in vacuo to provide 142.0 g of racemic 2-bromobutanoic acid, having a purity by GCA of 98%, ee of about 0% and yield of 85%.

### Synthesis Example 4

### Step 1: Preparation of (R)-2-Bromobutanoic Acid Chloride

A three-liter round bottomed flask fitted with stirrer, condenser, thermometer pocket, dropping funnel, nitrogen inlet and scrubber was flushed with nitrogen and charged with a solution of R-2-bromobutanoic acid (210.73 g) in toluene (210 g) solution with stirring. The solution was heated to about 48-50° C. To this, thionyl chloride (126.3 g) was added through the dropping funnel for 1.5 to 2 h at 48 to 50° C. Sulfur dioxide and hydrochloric acid gases evolved from the reaction were scrubbed into a sodium hydroxide aqueous solution. The reaction mass was heated at 60° C. until completion of the reaction, then concentrated under reduced pressure. R-2-bromobutanoic acid chloride in toluene solution (439 g) was obtained. Purity by GCA was 99.31%, ee was 95.1% and yield was 99% from R-2-bromobutanoic acid.

### Step 2: Preparation of (R)-2-Bromo-N-Benzylbutanamide

A three-liter round bottomed flask fitted with stirrer, condenser, thermometer pocket, dropping funnel and nitrogen inlet was charged with a solution of (R)-2-bromobutyric acid chloride (443.5 g) in toluene (744 g) with stirring. The solution was cooled to −2 to 3° C. To this solution benzylamine (118.5 g) was added through the dropping funnel for a 1 to 1.5 h period at −2-3° C. Sodium hydroxide aqueous solution (440 g) was then added dropwise for a 1-h period at −2-3° C. The reaction mass was stirred at −2-3° C. until completion of the reaction, then prepared for phase separation. The organic phase was separated. The aqueous phase was extracted with toluene and the organic phases were combined and washed with water. The combined organic phase was evaporated to dryness to provide the title compound (256 g). Purity by GCA was 98.74%, ee was 94% and yield was 98.7%.

### Step 3: Preparation of (2S)-N-benzyl-2-(4-fluoro-3-trifluoromethylphenoxy)-butanoic amide

A three-liter round bottomed flask fitted with stirrer, condenser, thermometer pocket, vacuum outlet and azeotrope water removal setup was charged with 4-fluoro-3-(trifluoromethyl)phenol (253.5 g), sodium hydroxide (100 g) and toluene (500 g) with stirring. The reaction mixture was heated to 55-60° C. and water was removed by azeotropic distillation under reduce pressure. Then a solution of R-2-bromo-N-benzyl butanamide (257 g) in toluene (500 g) was added to the reaction mixture at 50-55° C. The reaction mass was heated at 85-100° C. until completion of reaction. The reaction mixture was washed with dilute NaOH solution and the phases were separated. The aqueous phase was extracted with toluene. The combined organic phases were washed with brine solution. The brine-washed organic phase was treated for toluene recovery under reduced pressure until dryness. The resulting crude product was purified in isopropyl and water mixture. The title compound was obtained as a solid (317.51 g) with a purity of 99.6%, ee of 98.9% and yield of 88.5%.

What is claimed is:

1. A method for preparing compound S-1 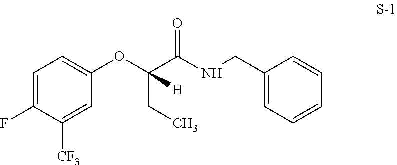 [SMILES: CC[C@H](Oc1ccc(F)c(C(F)(F)F)c1)C(=O)NCc1ccccc1]
from compound R-2 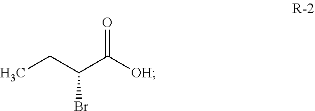
wherein compound R-2 is prepared by
treating compound rac-2 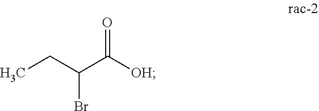
with a compound of Formula 3 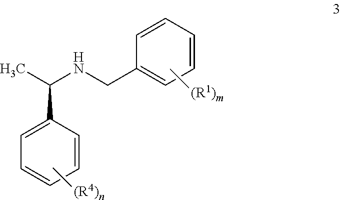
wherein
each R¹ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R²; or
two adjacent R¹ substituents are taken together with the phenyl to which they are attached to form a naphthalenyl ring optionally substituted with up to three R³;
each R² and each R³ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁴ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R⁵;
each R⁵ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
m is 0, 1, 2 or 3; and
n is 0, 1, 2 or 3;
to provide the R,R-salt of Formula 4 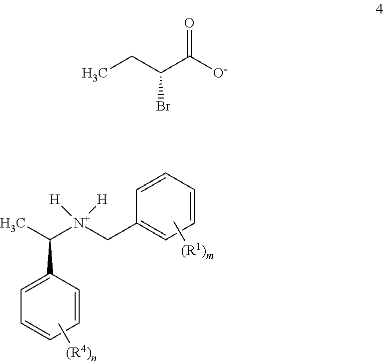
wherein R¹, R⁴, m and n are as defined for the compound of Formula 3;
selectively isolating the R,R-salt of Formula 4;
treating the R,R-salt of Formula 4 with a sodium base to provide compound R-5 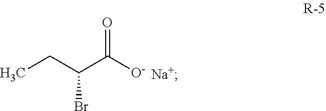
and
treating compound R-5 with acid.

2. The method of claim 1 wherein
m is 1 or 2;
n is 0; and
each R¹ is independently halogen, nitro, C₁-C₄ alkyl, C₁-C₄ haloalkyl or phenyl; or
two adjacent R¹ substituents are taken together with the phenyl to which they are attached to form an unsubstituted naphthalenyl ring.

3. The method of claim 1 wherein the compound of Formula 3 is selected from the group consisting of
(αR)-α-methyl-N-(phenylmethyl)-benzenemethanamine,
N-[(1R)-1-phenylethyl]-1-naphthalenemethanamine,
2,4-dichloro-N-[(1R)-1-phenylethyl]-benzenemethanamine,
3,4-dichloro-N-[(1R)-1-phenylethyl]-benzenemethanamine,
2,6-dichloro-N-[(1R)-1-phenylethyl]-benzenemethanamine,
2,4,6-trimethyl-N-[(1R)-1-phenylethyl]-benzenemethanamine,
4-nitro-N-[(1R)-1-phenylethyl]-benzenemethanamine, and
2-methyl-3-phenyl-N-[(1R)-1-phenylethyl]-benzenemethanamine.

4. The method of claim 3 wherein the compound of Formula 3 is N-[(1R)-1-phenylethyl]-1-naphthalenemethanamine.

5. The method of claim 1 wherein compound R-2 is converted to compound S-1 by the method comprising
treating compound R-2 with a C₁-C₆ alkanol to prepare the compound of Formula R-6; 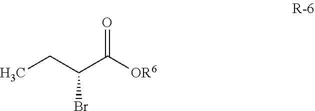
wherein R⁶ is C₁-C₆ alkyl;
treating the compound of Formula R-6 with compound 7 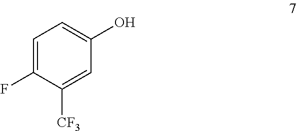 [SMILES: Oc1ccc(F)c(C(F)(F)F)c1]
to prepare the compound of Formula S-8 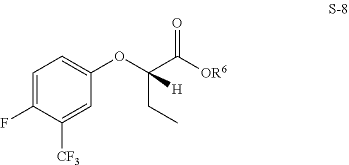
wherein OR⁴ is C₁-C₆ alkoxy;
and treating the compound of Formula S-8 with compound 9

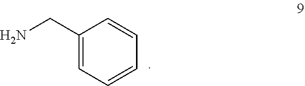

6. The method of claim 5 wherein OR⁴ is methoxy.

7. The method of claim 1 wherein compound R-2 is converted to compound S-1 by the method comprising
treating compound R-2 with a chlorinating agent to prepare compound R-10 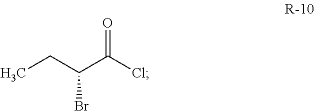
compound R-10 is treated with compound 9 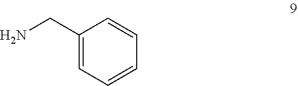
to prepare compound R-11 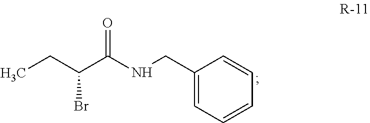
and
treating compound R-11 with compound 7

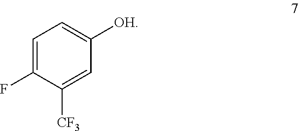 [SMILES: Oc1ccc(F)c(C(F)(F)F)c1]

8. A method for preparing compound S-1 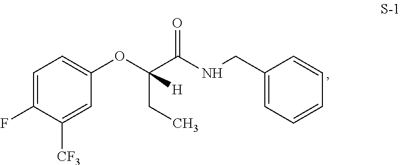 [SMILES: CC[C@H](Oc1ccc(F)c(C(F)(F)F)c1)C(=O)NCc1ccccc1]
the method comprising
preparing compound R-2 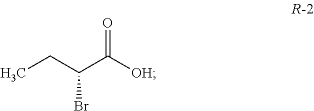
wherein compound R-2 is prepared by
treating compound rac-2 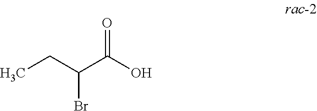
with a compound of Formula 3 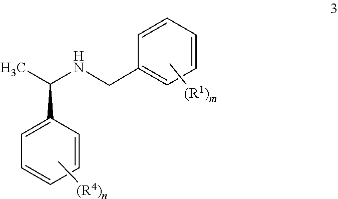
wherein
each R¹ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R²; or
two adjacent R¹ substituents are taken together with the phenyl to which they are attached to form a naphthalenyl ring optionally substituted with up to three R³;
each R² and each R³ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁴ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R⁵;
each R⁵ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
m is 0, 1, 2 or 3; and
n is 0, 1, 2 or 3;
to provide the R,R-salt of Formula 4 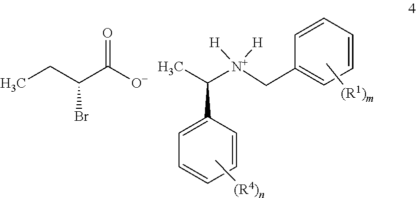
wherein R¹, R⁴, m and n are as defined for the compound of Formula 3;
selectively isolating the R,R-salt of Formula 4;
treating the R,R-salt of Formula 4 with a sodium base to provide compound R-5 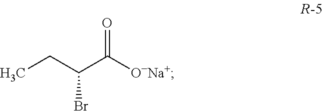
treating compound R-5 with acid; and
converting compound R-2 to the compound S-1.

9. The method of claim 8 wherein
m is 1 or 2;
n is 0; and
each R¹ is independently halogen, nitro, C₁-C₄ alkyl, C₁-C₄ haloalkyl or phenyl; or
two adjacent R¹ substituents are taken together with the phenyl to which they are attached to form an unsubstituted naphthalenyl ring.

10. The method of claim 8 wherein the compound of Formula 3 is selected from the group consisting of
(αR)-α-methyl-N-(phenylmethyl)-benzenemethanamine,
N-[(1R)-1-phenylethyl]-1-naphthalenemethanamine,
2,4-dichloro-N-[(1R)-1-phenylethyl]-benzenemethanamine,
3,4-dichloro-N-[(1R)-1-phenylethyl]-benzenemethanamine,
2,6-dichloro-N-[(1R)-1-phenylethyl]-benzenemethanamine,
2,4,6-trimethyl-N-[(1R)-1-phenylethyl]-benzenemethanamine,
4-nitro-N-[(1R)-1-phenylethyl]-benzenemethanamine, and
2-methyl-3-phenyl-N-[(1R)-1-phenylethyl]-benzenemethanamine.

11. The method of claim 10 wherein the compound of Formula 3 is N-[(1R)-1-phenylethyl]-1-naphthalenemethanamine.

12. The method of claim 8 wherein compound R-2 is converted to compound S-1 by the method comprising
treating compound R-2 with a C₁-C₆ alkanol to prepare the compound of Formula R-6; 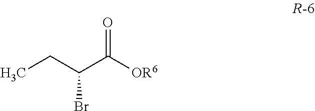
wherein OR⁶ is C₁-C₆ alkyl;
treating the compound of Formula R-6 with compound 7 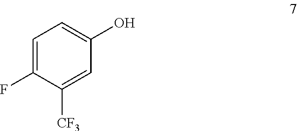 [SMILES: Oc1ccc(F)c(C(F)(F)F)c1]
to prepare the compound of Formula S-8 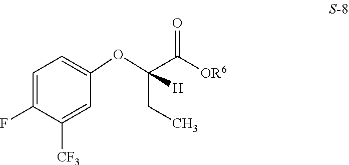
wherein R⁶ is C₁-C₆ alkyl;
and treating the compound of Formula S-8 with compound 9

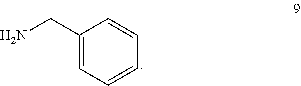

13. The method of claim 12 wherein R⁶ is methyl.

14. The method of claim 8 wherein compound R-2 is converted to compound S-1 by the method comprising
treating compound R-2 with a chlorinating agent to prepare compound R-10 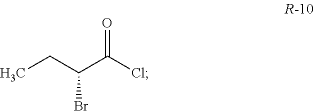
compound R-10 is treated with compound 9 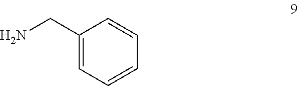
to prepare compound R-11 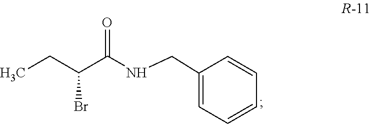
and
treating compound R-11 with compound 7

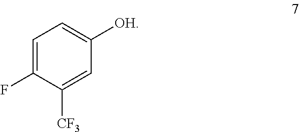 [SMILES: Oc1ccc(F)c(C(F)(F)F)c1]

15. A method for preparing compound S-1 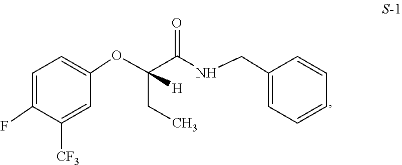 [SMILES: CC[C@H](Oc1ccc(F)c(C(F)(F)F)c1)C(=O)NCc1ccccc1]
the method comprising:
treating compound rac-2 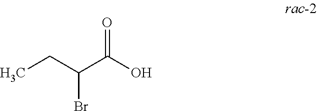
with a compound of Formula 3 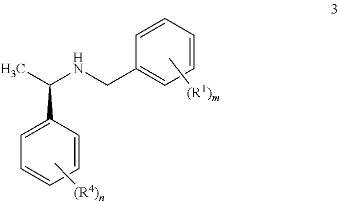
wherein
each R¹ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R²; or
two adjacent R¹ substituents are taken together with the phenyl to which they are attached to form a naphthalenyl ring optionally substituted with up to three R³;
each R² and each R³ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁴ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R⁵;
each R⁵ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
m is 0, 1, 2 or 3; and
n is 0, 1, 2 or 3;
to provide the R,R-salt of Formula 4 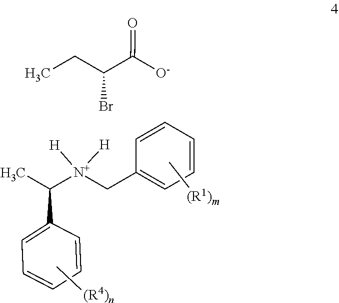
wherein R¹, R⁴, m and n are as defined for the compound of Formula 3;
selectively isolating the R,R-salt of Formula 4;
treating the R,R-salt of Formula 4 with a sodium base to provide compound R-5 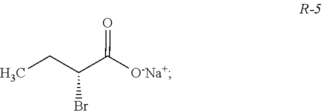
treating compound R-5 with acid to prepare compound R-2 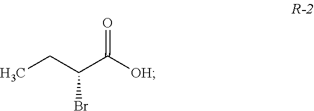
and
converting compound R-2 to compound S-1.

16. The method of claim 15 wherein
m is 1 or 2;
n is 0; and
each R¹ is independently halogen, nitro, C₁-C₄ alkyl, C₁-C₄ haloalkyl or phenyl; or
two adjacent R¹ substituents are taken together with the phenyl to which they are attached to form an unsubstituted naphthalenyl ring.

17. The method of claim 15 wherein the compound of Formula 3 is selected from the group consisting of
(αR)-α-methyl-N-(phenylmethyl)-benzenemethanamine,
N-[(1R)-1-phenylethyl]-1-naphthalenemethanamine,
2,4-dichloro-N-[(1R)-1-phenylethyl]-benzenemethanamine,
3,4-dichloro-N-[(1R)-1-phenylethyl]-benzenemethanamine,
2,6-dichloro-N-[(1R)-1-phenylethyl]-benzenemethanamine,
2,4,6-trimethyl-N-[(1R)-1-phenylethyl]-benzenemethanamine,
4-nitro-N-[(1R)-1-phenylethyl]-benzenemethanamine, and
2-methyl-3-phenyl-N-[(1R)-1-phenylethyl]-benzenemethanamine.

18. The method of claim 17 wherein the compound of Formula 3 is N-[(1R)-1-phenylethyl]-1-naphthalenemethanamine.

19. The method of claim 15 wherein compound R-2 is converted to compound S-1 by the method comprising
treating compound R-2 with a C₁-C₆ alkanol to prepare the compound of Formula R-6; 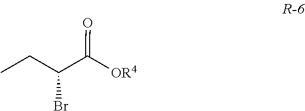
wherein OR⁴ is C₁-C₆ alkoxy;
treating the compound of Formula R-6 with the compound of Formula 7 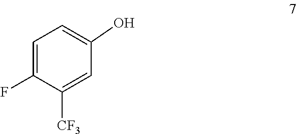 [SMILES: Oc1ccc(F)c(C(F)(F)F)c1]
to prepare the compound of Formula S-8 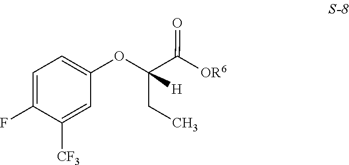
wherein R⁶ is C₁-C₆ alkyl;
and treating the compound of Formula S-8 with compound 9

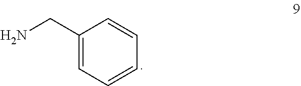

20. The method of claim 19 wherein R⁶ is methyl.

21. The method of claim 15 wherein compound R-2 is converted to compound S-1 by the method comprising
treating compound R-2 with a chlorinating agent to prepare compound R-10 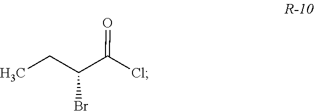
compound R-10 is treated with compound 9 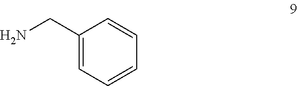
to prepare compound R-11 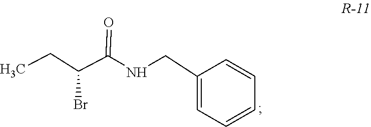
and
treating compound R-11 with compound 7

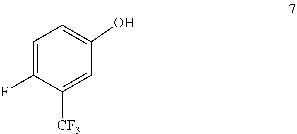 [SMILES: Oc1ccc(F)c(C(F)(F)F)c1]

22. A method for preparing compound S-1 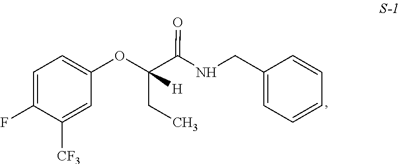 [SMILES: CC[C@H](Oc1ccc(F)c(C(F)(F)F)c1)C(=O)NCc1ccccc1]
the method comprising:
treating compound rac-2 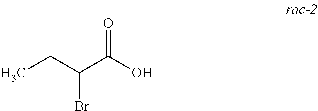
with a compound of Formula 3 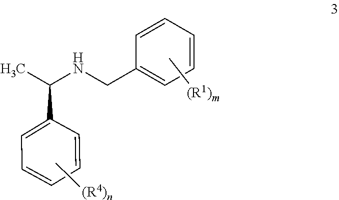
wherein
each R¹ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R²; or
two adjacent R¹ substituents are taken together with the phenyl to which they are attached to form a naphthalenyl ring optionally substituted with up to three R³;
each R² and each R³ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁴ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R⁵;
each R⁵ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
m is 0, 1, 2 or 3; and
n is 0, 1, 2 or 3;
to provide the R,R-salt of Formula 4 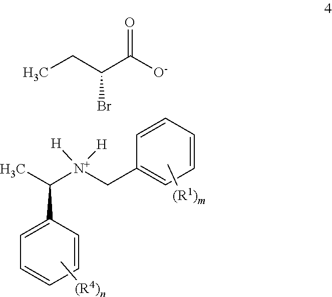
wherein R¹, R⁴, m and n are as defined for the compound of Formula 3;
selectively isolating the R,R-salt of Formula 4;
treating the R,R-salt of Formula 4 with a sodium base to provide compound R-5 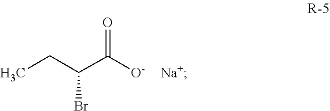
treating compound R-5 with acid to prepare compound R-2 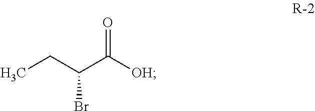
treating compound R-2 with a chlorinating agent to prepare compound R-10 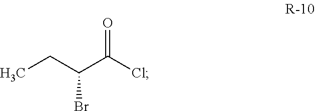
treating compound R-10 with compound 9 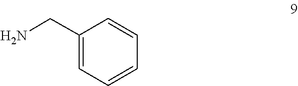
to prepare compound R-11 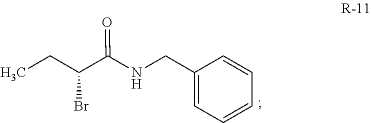
treating compound R-11 with compound 7

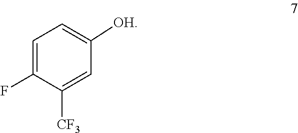 [SMILES: Oc1ccc(F)c(C(F)(F)F)c1]

23. The method of claim 22 wherein
m is 1 or 2;
n is 0; and
each R¹ is independently halogen, nitro, C₁-C₄ alkyl, C₁-C₄ haloalkyl or phenyl; or
two adjacent R¹ substituents are taken together with the phenyl to which they are attached to form an unsubstituted naphthalenyl ring.

24. The method of claim 22 wherein the compound of Formula 3 is selected from the group consisting of
(αR)-α-methyl-N-(phenylmethyl)-benzenemethanamine,
N-[(1R)-1-phenylethyl]-1-naphthalenemethanamine,
2,4-dichloro-N-[(1R)-1-phenylethyl]-benzenemethanamine,
3,4-dichloro-N-[(1R)-1-phenylethyl]-benzenemethanamine,
2,6-dichloro-N-[(1R)-1-phenylethyl]-benzenemethanamine,
2,4,6-trimethyl-N-[(1R)-1-phenylethyl]-benzenemethanamine,
4-nitro-N-[(1R)-1-phenylethyl]-benzenemethanamine, and
2-methyl-3-phenyl-N-[(1R)-1-phenylethyl]-benzenemethanamine.

25. The method of claim 24 wherein the compound of Formula 3 is N-[(1R)-1-phenylethyl]-1-naphthalenemethanamine.

26. A method for preparing compound R-2 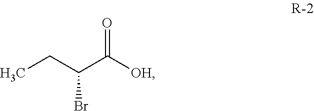
the method comprising:
treating compound rac-2 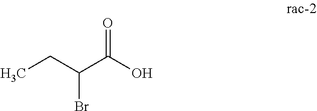
with a compound of Formula 3 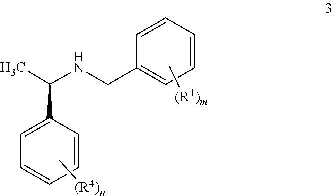
wherein
each R¹ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R²; or
two adjacent R¹ substituents are taken together with the phenyl to which they are attached to form a naphthalenyl ring optionally substituted with up to three R³;
each R² and each R³ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁴ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R⁵;
each R⁵ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
m is 0, 1, 2 or 3; and
n is 0, 1, 2 or 3;
to provide the R,R-salt of Formula 4 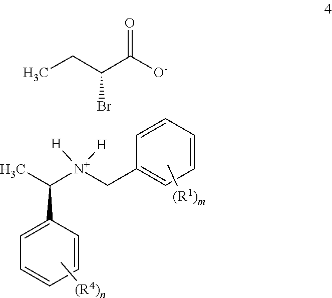
wherein R¹, R⁴, m and n are as defined for the compound of Formula 3;
selectively isolating the R,R-salt of Formula 4;
treating the R,R-salt of Formula 4 with a sodium base to provide compound R-5 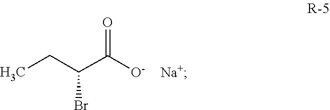
treating compound R-5 with acid.

27. The method of claim 26 wherein
m is 1 or 2;
n is 0; and
each R¹ is independently halogen, nitro, C₁-C₄ alkyl, C₁-C₄ haloalkyl or phenyl; or
two adjacent R¹ substituents are taken together with the phenyl to which they are attached to form an unsubstituted naphthalenyl ring.

28. The method of claim 26 wherein the compound of Formula 3 is selected from the group consisting of
(αR)-α-methyl-N-(phenylmethyl)-benzenemethanamine,
N-[(1R)-1-phenylethyl]-1-naphthalenemethanamine,
2,4-dichloro-N-[(1R)-1-phenylethyl]-benzenemethanamine,
3,4-dichloro-N-[(1R)-1-phenylethyl]-benzenemethanamine,
2,6-dichloro-N-[(1R)-1-phenylethyl]-benzenemethanamine,
2,4,6-trimethyl-N-[(1R)-1-phenylethyl]-benzenemethanamine,
4-nitro-N-[(1R)-1-phenylethyl]-benzenemethanamine, and
2-methyl-3-phenyl-N-[(1R)-1-phenylethyl]-benzenemethanamine.

28. The method of claim 27 wherein the compound of Formula 3 is N-[(1R)-1-phenylethyl]-1-naphthalenemethanamine.

29. A method for preparing compound rac-2 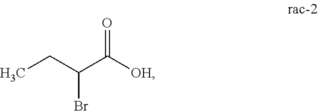
the method comprising:
treating the enantiomerically enriched compound of Formula scal-2 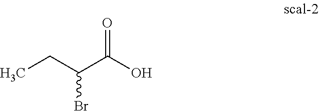
with hydrobromic acid or a quaternary ammonium bromide salt.

30. The method of claim 29 wherein compound scal-2 is predominantly (S)-2-bromobutanoic acid.

31. An R,R-salt of Formula 4 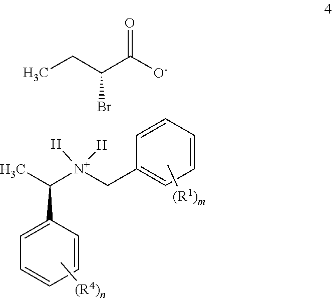
wherein
each R¹ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R²; or
two adjacent R¹ substituents are taken together with the phenyl to which they are attached to form a naphthalenyl ring optionally substituted with up to three R³;
each R² and each R³ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
each R⁴ is independently halogen, nitro, cyano, C₁-C₆ alkyl, C₁-C₆ haloalkyl, C₁-C₆ alkoxy, C₁-C₆ alkenyl, C₁-C₆ haloalkenyl, C₁-C₆ haloalkoxy; or phenyl optionally substituted with up to two R⁵;
each R⁵ is independently halogen, nitro, cyano, C₁-C₆ alkyl, C₁-C₆ haloalkyl, C₁-C₆ alkoxy, C₁-C₆ alkenyl, C₁-C₆ haloalkenyl or C₁-C₆ haloalkoxy;
m is 0, 1, 2 or 3; and
n is 0, 1, 2 or 3.

32. The R,R-salt of claim 31 wherein
m is 1 or 2;
n is 0; and
each R¹ is independently halogen, nitro, C₁-C₄ alkyl, C₁-C₄ haloalkyl or phenyl; or
two adjacent R¹ substituents are taken together with the phenyl to which they are attached to form an unsubstituted naphthalenyl ring.

33. The R,R-salt of claim 31 comprising the salt of an amine selected from the group consisting of
(αR)-α-methyl-N-(phenylmethyl)-benzenemethanamine,
N-[(1R)-1-phenylethyl]-1-naphthalenemethanamine,
2,4-dichloro-N-[(1R)-1-phenylethyl]-benzenemethanamine,
3,4-dichloro-N-[(1R)-1-phenylethyl]-benzenemethanamine,
2,6-dichloro-N-[(1R)-1-phenylethyl]-benzenemethanamine,
2,4,6-trimethyl-N-[(1R)-1-phenylethyl]-benzenemethanamine,
4-nitro-N-[(1R)-1-phenylethyl]-benzenemethanamine, and
2-methyl-3-phenyl-N-[(1R)-1-phenylethyl]-benzenemethanamine.

34. The R,R-salt of claim 33 comprising the salt of N-[(1R)-1-phenylethyl]-1-naphthalenemethanamine.
